(19) United States
(12) Patent Application Publication
(10) Pub. No.: US 2025/0042906 A1
(43) Pub. Date: Feb. 6, 2025

(54) PROCESSES FOR MAKING IRAK4 INHIBITORS

(71) Applicant: Gilead Sciences, Inc., Foster City, CA (US)

(72) Inventors: Stephen E. AMMANN, Foster City, CA (US); Angie R. ANGELES, San Diego, CA (US); Matthew R. CHIN, Danvers, MA (US); Jeromy J. COTTELL, Redwood City, CA (US); Ian J. DOXSEE, San Mateo, CA (US); Peter C. FUNG, San Mateo, CA (US); Brittanie T. HOANG, New York, NY (US); Stephen LAU, South San Francisco, CA (US); Devon A. MUNDAL, Seattle, WA (US); Sean T. NEVILLE, San Mateo, CA (US); John REICHWEIN, Bedminster, NJ (US); Erik RUEDEN, San Mateo, CA (US); May G. YOUNG, Fremont, CA (US); Chia-Yun YU, San Mateo, CA (US)

(21) Appl. No.: 18/758,879

(22) Filed: Jun. 28, 2024

Related U.S. Application Data
(60) Provisional application No. 63/511,558, filed on Jun. 30, 2023.

(51) Int. Cl. C07D 487/04

(52) U.S. Cl. CPC C07D 487/04

(57) ABSTRACT

The present disclosure relates generally to processes for preparing compounds that are inhibitors of the kinase IRAK4 and the synthetic intermediates prepared thereby.

### CROSS-REFERENCE TO RELATED APPLICATIONS

This application claims the benefit under 35 U.S.C. § 119 (c) of U.S. Provisional Application Ser. No. 63/511,558, filed Jun. 30, 2023, the contents of which are hereby incorporated by reference in its entirety.

### FIELD

The present disclosure relates generally to processes for preparing compounds that are inhibitors of the kinase IRAK4 and the synthetic intermediates prepared thereby.

### BACKGROUND

Interleukin-1 receptor-associated kinase-4 (IRAK4) is a serine-threonine kinase which acts as a mediator in interleukin-1/Toll-like receptor (IL-1/TLR) signaling cascades. More particularly, IRAK4 is involved in activation of adaptor protein myeloid differentiation primary response gene 88 (MyD88) signaling cascades and is hypothesized to play a role in inflammatory and fibrotic disorders, such as rheumatoid arthritis (RA), inflammatory bowel disease (IBD), gout. Lyme disease, arthritis, psoriasis, pelvic inflammatory disease, systemic lupus erythematosus (SLE), Sjogren's syndrome, viral myocarditis, acute and chronic tissue injury, non-alcoholic steatohepatitis (NASH), alcoholic hepatitis and kidney disease, including chronic kidney disease and diabetic kidney disease. In addition, IRAK4 plays a role in certain cancers and is hypothesized to play a role in inflammation associated with gastrointestinal infections, including C. difficile. Signaling through IL-IR/TLR results in the activation of MyD88 which recruits IRAK4 and IRAK1 to form a signaling complex. This complex then interacts with a series of kinases, adaptor proteins, and ligases, ultimately resulting in the activation of nuclear factor kappa-light-chain-enhancer of activated B cells (NF-κB), activator protein-1 (API), cyclic AMP-responsive element-binding protein (CREB) and the interferon-regulatory factors (IRFs), including IRF5 and IRF7, inducing the generation of pro-inflammatory cytokines and type I interferons.

Therefore, inhibitors of IRAK4 may be useful in the treatment of inflammatory and fibrotic disorders, such as rheumatoid arthritis (RA), inflammatory bowel disease (IBD), gout. Lyme disease, arthritis, psoriasis, pelvic inflammatory disease, systemic lupus erythematosus (SLE), Sjogren's syndrome, inflammation associated with gastrointestinal infections, including C. difficile, viral myocarditis, acute and chronic tissue injury, non-alcoholic steatohepatitis (NASH), alcoholic hepatitis, and kidney disease, including chronic kidney disease and diabetic kidney disease.

### SUMMARY

Provided herein is a process for preparing Compound I, or a salt thereof:

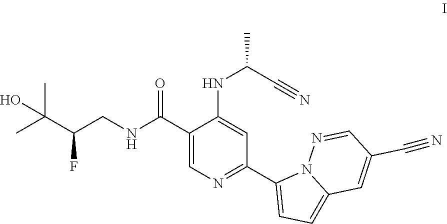 [SMILES: C[C@H](C#N)Nc1cc(-c2ccc3cc(C#N)cnn23)ncc1C(=O)NC[C@@H](F)C(C)(C)O]

comprising contacting Compound II, or a salt thereof;

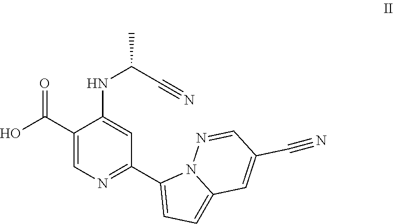 [SMILES: C[C@H](C#N)Nc1cc(-c2ccc3cc(C#N)cnn23)ncc1C(=O)O]

with Compound II, or a salt thereof:

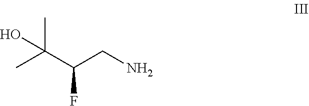

under conditions suitable to provide Compound I.

Also provided is a citric acid salt of Compound I:

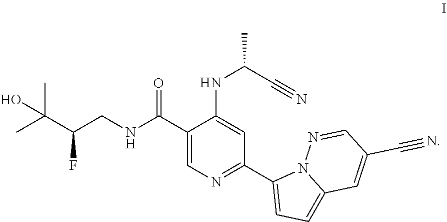 [SMILES: C[C@H](C#N)Nc1cc(-c2ccc3cc(C#N)cnn23)ncc1C(=O)NC[C@@H](F)C(C)(C)O]

Also provided is a methanesulfonic acid salt of Compound II:

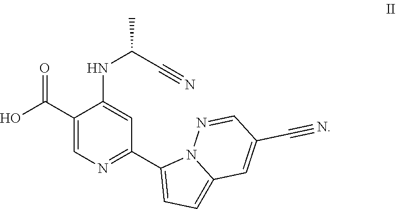 [SMILES: C[C@H](C#N)Nc1cc(-c2ccc3cc(C#N)cnn23)ncc1C(=O)O]

Also provided is a process for preparing Compound II, or a salt thereof:

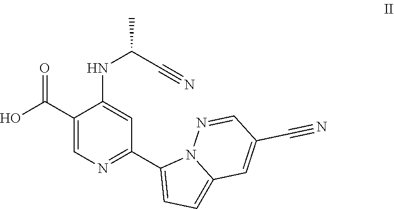 [SMILES: C[C@H](C#N)Nc1cc(-c2ccc3cc(C#N)cnn23)ncc1C(=O)O]

comprising contacting a compound of Formula IV, or a salt thereof;

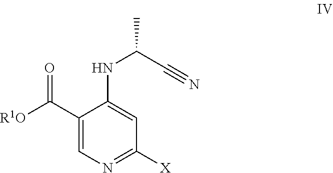

with a compound of Formula V, or a salt thereof:

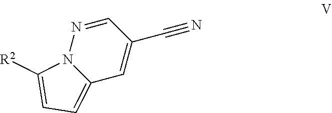

under conditions suitable to provide Compound I; wherein:
X is halo;
R¹ is a protecting group;
R² is hydrogen or —B(OR³)₂; and
each R³ is independently hydrogen or C₁₋₆ alkyl, or two R³ cyclize to form a cyclic boronate ester.

Also provided is a process for preparing Compound I, or a salt thereof:

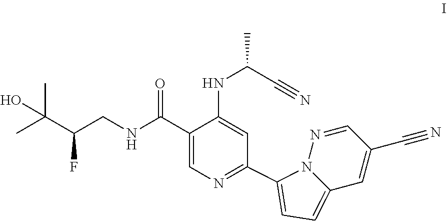 [SMILES: C[C@H](C#N)Nc1cc(-c2ccc3cc(C#N)cnn23)ncc1C(=O)NC[C@@H](F)C(C)(C)O]

comprising contacting compound of Formula VI, or a salt thereof:

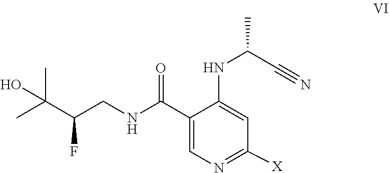

with a compound of Formula V, or a salt thereof:

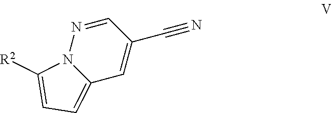

under conditions suitable to provide Compound I; provided the conditions do not comprise microwave radiation; wherein:
X is halo;
R² is hydrogen or —B(OR³)₂; and
each R³ is independently hydrogen or C₁₋₆ alkyl, or two R³ cyclize to form a cyclic boronate ester.

Also provided is a process for preparing a compound of Formula VI, or a salt thereof:

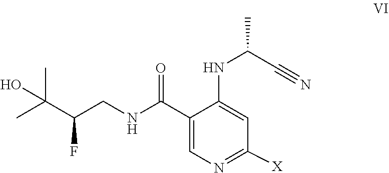

comprising contacting a compound of Formula VII, or a salt thereof;

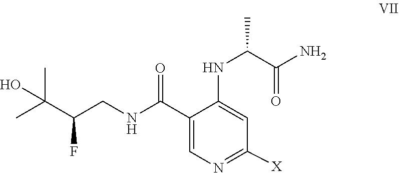

with trifluoroacetic anhydride in the absence of an organic base, under conditions suitable to provide a compound of Formula VIII:

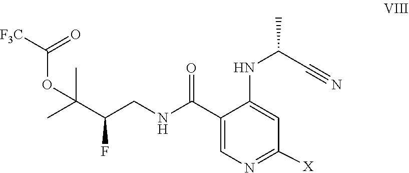

wherein X is halo; and hydrolyzing a compound of Formula VIII to provide a compound of Formula VI.

Also provided is a process for preparing a compound of Formula VI, or a salt thereof:

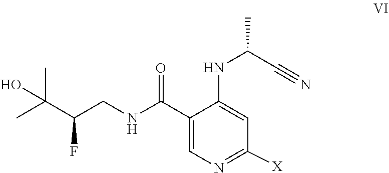

comprising contacting a compound of Formula VII, or a salt thereof;

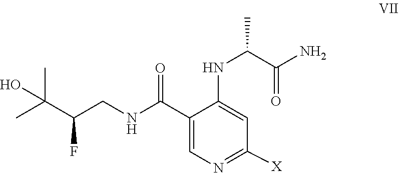

with a dehydrating agent under conditions suitable to provide a compound of Formula VI; wherein X is halo.

In some embodiments, the dehydrating agent is other than trifluoroacetic anhydride.

Also provided is a process for preparing a compound of Formula IX, or a salt thereof:

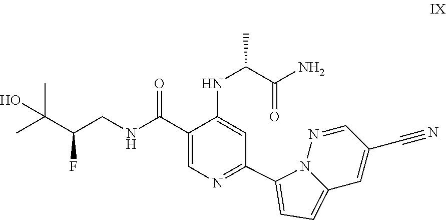 [SMILES: C[C@@H](Nc1cc(-c2ccc3cc(C#N)cnn23)ncc1C(=O)NC[C@@H](F)C(C)(C)O)C(N)=O]

comprising contacting a compound of Formula VII, or a salt thereof;

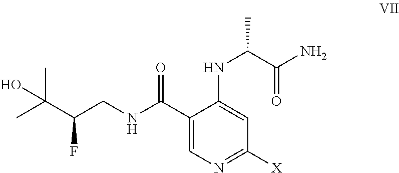

with a compound of Formula V, or a salt thereof:

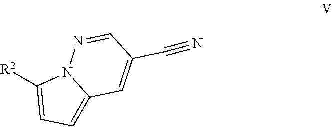

under conditions suitable to provide Compound IX; wherein:
X is halo;
R² is hydrogen or —B(OR³)₂; and
each R³ is independently hydrogen or C₁₋₆ alkyl, or two R³ cyclize to form a cyclic boronate ester.

Also provided is a process for preparing Compound I, or a salt thereof:

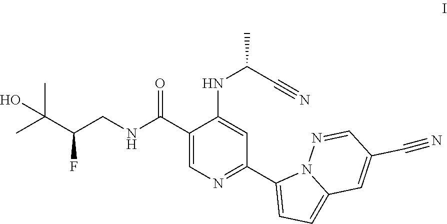 [SMILES: C[C@H](C#N)Nc1cc(-c2ccc3cc(C#N)cnn23)ncc1C(=O)NC[C@@H](F)C(C)(C)O]

comprising contacting a compound of Formula IX, or a salt thereof;

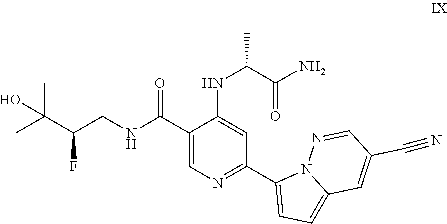 [SMILES: C[C@@H](Nc1cc(-c2ccc3cc(C#N)cnn23)ncc1C(=O)NC[C@@H](F)C(C)(C)O)C(N)=O]

under conditions suitable to provide Compound I.

Also provided is a compound, or a salt thereof, selected from:

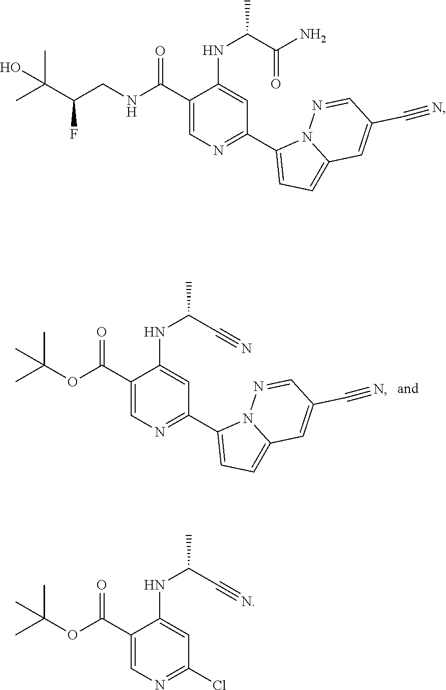 [SMILES: C[C@@H](Nc1cc(-c2ccc3cc(C#N)cnn23)ncc1C(=O)NC[C@@H](F)C(C)(C)O)C(N)=O ; C[C@H](C#N)Nc1cc(-c2ccc3cc(C#N)cnn23)ncc1C(=O)OC(C)(C)C ; C[C@H](C#N)Nc1cc(Cl)ncc1C(=O)OC(C)(C)C]

### DETAILED DESCRIPTION

### Definitions

As used in the present specification, the following words and phrases are generally intended to have the meanings as set forth below, except to the extent that the context in which they are used indicates otherwise.

The term “comprise” and variations thereof, such as, “comprises” and “comprising” are to be construed in an open, inclusive sense, that is, as “including, but not limited to.” Further, the singular forms “a,” “an,” and “the” include plural references unless the context clearly dictates otherwise. Thus, reference to “the compound” includes a plurality of such compounds, and reference to “the assay” includes reference to one or more assays and equivalents thereof known to those skilled in the art.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±2.5%. In certain other embodiments, the term “about” includes the indicated amount ±1%. Also, to the term “about X” includes description of “X”.

Recitation of numeric ranges of values throughout the disclosure is intended to serve as a shorthand notation of referring individually to each separate value falling within the range inclusive of the values defining the range, and each separate value is incorporated in the specification as it were individually recited herein.

As used herein, the term “contacting” refers to the process of bringing into contact at least two distinct species such that they can react. It should be appreciated, however, that the resulting reaction product can be produced directly from a reaction between the added reagents or from an intermediate from one or more of the added reagents which can be produced in the reaction mixture.

The term “reaction conditions” is intended to refer to the physical and/or environmental conditions under which a chemical reaction proceeds. Examples of reaction conditions include, but are not limited to, one or more of following: reaction temperature, solvent, pH, pressure, reaction time, mole ratio of reactants, the presence of a base or acid, one or more protecting groups, or catalyst, radiation, etc. Reaction conditions may be named after the particular chemical reaction in which the conditions are employed, such as, coupling conditions, hydrogenation conditions, acylation conditions, reduction conditions, etc. Reaction conditions for most reactions are generally known to those skilled in the art or can be readily obtained from the literature. Exemplary reaction conditions sufficient for performing the chemical transformations provided herein can be found throughout, and in particular, the examples below. It is also contemplated that the reaction conditions can include reagents in addition to those listed in the specific reaction.

As used herein, “under conditions suitable” is intended to refer to the reaction conditions under which the desired chemical reaction may proceed.

“Protecting group” refers to a moiety of a compound that masks or alters the properties of a functional moiety. “Deprotecting” or “deprotection” refers to a step removing the protecting group so as to restore the functional moiety to its original state. Chemical protecting groups and strategies for protection/deprotection are well known in the art. See also Protective Groups in Organic Chemistry, Peter G. M. Wuts and Theodora W. Greene, 4th Ed., 2006. Protecting groups are often utilized to mask the reactivity of certain functional moieties, to assist in the efficiency of desired chemical reactions, e.g., making and breaking chemical bonds in an ordered and planned fashion. For example, a “carboxylic acid protecting group” refers to a protecting group useful for masking the carboxylic acid moiety, e.g., to render the carboxylic acid group unreactive during intermediate steps of a synthetic process. Exemplary carboxylic acid protecting groups include alkyl protecting groups, such as methyl, ethyl, benzyl, or tert-butyl; silyl groups such as 2-(trimethylsilyl)ethyl; and thioesters such as tert-butyl thioester. In some embodiments, the protecting group is tert-butyl.

“Catalyst” refers to a chemical reactant that increases the rate of a reaction without itself being consumed. Similarly, a “precatalyst” refers to a compound that is converted to a catalyst during the course of the catalyzed reaction.

A “dehydrating agent” as used herein is a chemical reactant that is capable of converting an amide group to a nitrile group through the removal of water. Exemplary dehydrating agents include cyanuric chloride, anhydrides such as acetic anhydride, trifluoromethanesulfonic acid anhydride, or trifluoroacetic anhydride, ethyl chloroformate, phosphorus oxychloride, and phosphorus pentoxide.

“Hydrolyzing” or “hydrolysis” as used herein refers to the cleavage by water of a carboxylic ester into a carboxylic acid and an alcohol. Exemplary hydrolyzing agents include inorganic bases such as sodium carbonate, sodium bicarbonate, potassium carbonate, potassium bicarbonate, and alkoxide bases such as sodium methoxide and potassium methoxide.

A “ligand” as used herein refers to an ion or molecule that binds to a metal atom to form a coordination complex. Denticity of a ligand refers to the number of times a ligand bonds to a metal through noncontiguous donor sites. Ligands may be monodentate (i.e., they possess one binding site) or bidentate (i.e., they possess two binding sites).

As used herein, the term “salt” refers to a compound formed by the reaction of an acid and a base, resulting in the formation of a positively charged cation and a negatively charged anion. In general, a salt is defined as a compound that is formed by the combination of positively and negatively charged ions, where the charges of the ions result in a neutral compound. Salts can be either inorganic or organic. As used herein, the term “salt” includes partially or fully ionized salt forms. In some embodiments, the salt is fully ionized.

Definitions of specific functional groups and chemical terms are described in more detail below. For purposes of this disclosure, the chemical elements are identified in accordance with the Periodic Table of the Elements. CAS version. Handbook of Chemistry and Physics. 75th Ed., inside cover, and specific functional groups are generally defined as described therein. Additionally, general principles of organic chemistry, as well as specific functional moieties and reactivity, are described in Organic Chemistry, Thomas Sorrell. University Science Books, Sausalito, 1999: Smith and March March's Advanced Organic Chemistry, 5th Edition. John Wiley & Sons. Inc., New York, 2001; Larock, Comprehensive Organic Transformations, VCH Publishers, Inc., New York. 1989; Carruthers, Some Modern Methods of Organic Synthesis, 3rd Edition, Cambridge University Press, Cambridge. 1987; the entire contents of each of which are incorporated herein by reference.

The term “alkyl” as used herein, means a straight or branched, saturated hydrocarbon chain containing from 1 to 30 carbon atoms. The term “lower alkyl” or “C₁-C₆-alkyl” means a straight or branched chain hydrocarbon containing from 1 to 6 carbon atoms. The term “C₁-C₃-alkyl” means a straight or branched chain hydrocarbon containing from 1 to 3 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

“Alkoxy” refers to the group “alkyl-O—”. Examples of alkoxy groups include methoxy, ethoxy, n-propoxy, iso-propoxy, n-butoxy, tert-butoxy, sec-butoxy, n-pentoxy, n-hexoxy, and 1,2-dimethylbutoxy.

The term “aryl” as used herein, refers to a phenyl group, or bicyclic aryl or tricyclic aryl fused ring systems. Bicyclic fused ring systems are exemplified by a phenyl group appended to the parent molecular moiety and fused to a phenyl group. Tricyclic fused ring systems are exemplified by a phenyl group appended to the parent molecular moiety and fused to two other phenyl groups. Representative examples of bicyclic aryls include, but are not limited to, naphthyl. Representative examples of tricyclic aryls include, but are not limited to, anthracenyl. The monocyclic, bicyclic, and tricyclic aryls are connected to the parent molecular moiety through any carbon atom contained within the rings, and can be unsubstituted or substituted.

The term “halogen” or “halo” as used herein, means Cl, Br, I, or F.

The term “amine base” as used herein generally refers to a primary, secondary, or tertiary amine, such as a alkyl amine, dialkyl amine, trialkyl amine, nitrogen-containing heterocycle, or nitrogen-containing heteroaryl, wherein each of which is optionally substituted, e.g., by alkyl.

The term “nitrogen-containing heterocyclic” base as used herein, refers to a saturated or partially unsaturated cyclic alkyl group, with at least one ring nitrogen atom, and optionally one or more additional ring heteroatoms independently selected from nitrogen, oxygen, and sulfur. The term “heterocyclic” includes heterocycloalkenyl rings (i.e., a heterocyclic ring having at least one double bond), bridged-heterocyclic rings, fused-heterocyclic rings, and spiro-heterocyclic rings. A heterocycle may be a single ring or multiple rings wherein the multiple rings may be fused, bridged or spiro, and may comprise one or more (e.g., one to three or one or two) oxo (═O) or N-oxide (—O⁻) moieties. Any non-aromatic ring containing at least one nitrogen atom capable of accepting a proton is considered a nitrogen-containing heterocyclic base. Further, the term heterocyclic is intended to encompass any non-aromatic ring containing at least one heteroatom, which ring may be fused to a cycloalkyl, aryl or heteroaryl ring, regardless of the position of the heteroatom. As used herein, a nitrogen-containing heterocyclic has 2 to 20 ring carbon atoms (i.e. C₂₋₂₀ heterocycle), 2 to 12 ring carbon atoms (i.e., C₂₋₁₂ heterocycle), 2 to 10 ring carbon atoms (i.e., C₂₋₁₀ heterocycle), 2 to 8 ring carbon atoms (i.e., C₂₋₈ heterocycle), 3 to 12 ring carbon atoms (i.e., C₃₋₁₂ heterocycle), 3 to 8 ring carbon atoms (i.e., C₃₋₈ heterocycle), or 3 to 6 ring carbon atoms (i.e., C₃₋₆ heterocycle); having at least 1 nitrogen atom capable of accepting a proton, and optionally an additional 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen.

The term “nitrogen-containing heteroaryl” base refers to an aromatic group having a single ring, multiple rings or multiple fused rings, with at least one nitrogen atom capable of accepting a proton, and optionally one or more additional ring heteroatoms independently selected from nitrogen, oxygen and sulfur. As used herein, nitrogen-containing heteroaryl includes 1 to 20 ring carbon atoms (i.e., C₁₋₂₀) heteroaryl), 3 to 12 ring carbon atoms (i.e., C₃₋₁₂ heteroaryl), or 3 to 8 carbon ring atoms (i.e., C₃₋₈ heteroaryl); and having at least 1 nitrogen atom, and optionally an additional 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are embraced by the present disclosure and are disclosed herein just as if each and every combination was individually and explicitly disclosed, to the extent that such combinations embrace subject matter that are, for example, compounds that are stable compounds (i.e., compounds that can be made, isolated, characterized, and tested for biological activity). In addition, all sub-combinations of the various embodiments and elements thereof (e.g., elements of the chemical groups listed in the embodiments describing such variables) are also embraced by the present disclosure and are disclosed herein just as if each and every such sub-combination was individually and explicitly disclosed herein.

### Processes

The processes described herein provide compounds that are inhibitors of the kinase IRAK4.

Provided herein is a process for preparing Compound I, or a salt thereof:

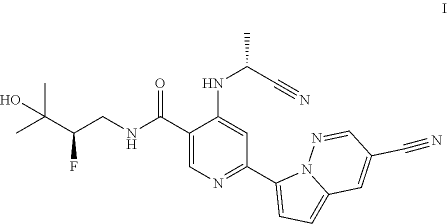 [SMILES: C[C@H](C#N)Nc1cc(-c2ccc3cc(C#N)cnn23)ncc1C(=O)NC[C@@H](F)C(C)(C)O]

comprising contacting Compound II, or a salt thereof:

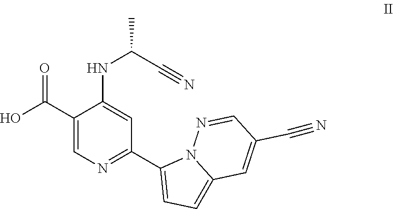 [SMILES: C[C@H](C#N)Nc1cc(-c2ccc3cc(C#N)cnn23)ncc1C(=O)O]

with Compound III, or a salt thereof:

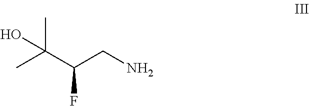

under conditions suitable to provide Compound I.

In some embodiments, the conditions comprise a coupling agent and a base.

In some embodiments, the base is an amine base or a carbonate base. In some embodiments, the base is N-methylimidazole, N,N-diisopropylethylamine, triethylamine, N-methylmorpholine, potassium carbonate, or sodium carbonate.

In some embodiments, the base is an amine. In some embodiments, the amine is N-methylimidazole, N,N-diisopropylethylamine, triethylamine, or N-methylmorpholine. In some embodiments, the base is N-methylimidazole. In some embodiments, the base is N,N-diisopropylethylamine.

In some embodiments, the base is a carbonate base. In some embodiments, the carbonate base is potassium carbonate, or sodium carbonate.

Suitable coupling agents (or activating agents) are known in the art and include for example, carbonyl diimidazole (e.g., N,N′-dicyclohexylcarbodiimide (DCC), N,N-dicyclopentylcarbodiimide, N,N′-diisopropylcarbodiimide (DIC), 1-ethyl-3-(3-dimethylaminopropyl) carbodiimide (EDC), N-t-butyl-N-methylcarbodiimide (BMC), N-t-butyl-N-ethylcarbodiimide (BEC), 1,3-bis(2,2-dimethyl-1,3-dioxolan-4-ylmethyl) carbodiimide (BDDC), etc.), anhydrides (e.g., symmetric, mixed, or cyclic anhydrides), activated ester forming agents (e.g., phenyl activated ester derivatives, p-hydroxamic activated ester, hexafluoroacetone (HFA), etc.), acylazole forming agents (acylimidazoles using CDI, acylbenzotriazoles, etc.), acyl azide forming agents, acid halide forming agents, phosphonium salts (HOBt, PyBOP, HOAt, etc.), aminium/uronium salts (e.g., tetramethyl aminium salts, bispyrrolidino aminium salts, bispiperidino aminium salts, imidazolium uronium salts, pyrimidinium uronium salts, uronium salts derived from N,N,N′-trimethyl-N′-phenylurea, morpholino-based aminium/uronium coupling reagents, antimoniate uronium salts, etc.), organophosphorus reagents (e.g., phosphinic and phosphoric acid derivatives), organosulfur reagents (e.g., sulfonic acid derivatives), triazine coupling reagents (e.g., 2-chloro-4,6-dimethoxy-1,3,5-triazine, 4-(4,6-dimethoxy-1,3,5-triazin-2-yl)-4 methylmorpholinium chloride, 4-(4,6-dimethoxy-1,3,5-triazin-2-yl)-4 methylmorpholinium tetrafluoroborate, etc.), pyridinium coupling reagents (e.g., Mukaiyama's reagent, pyridinium tetrafluoroborate coupling reagents, etc.), polymer-supported reagents (e.g., polymer-bound carbodiimide, polymer-bound TBTU, polymer-bound 2,4,6-trichloro-1,3,5-triazine, polymer-bound HOBt, polymer-bound HOSu, polymer-bound IIDQ, polymer-bound EEDQ, etc.), and the like.

In some embodiments, the coupling agent provides an acid halide intermediate, such as an acid chloride. In some embodiments, the coupling agent is thionyl chloride, oxalyl chloride, or phosphorus (V) oxychloride.

In some embodiments, the coupling agent provides a mixed anhydride intermediate. In some embodiments, the coupling agent is acetic anhydride, pivaloyl chloride, diphenylphosphinic anhydride, propanephosphinic anhydride, boric acid, isobutyl chloroformate, methanesulfonyl chloride, or p-toluenesulfonyl chloride.

In some embodiments, the coupling agent provides an activated ester. In some embodiments, the coupling agent is 4-(4,6-dimethoxy-1,3,5-triazin-2-yl)-4-methylmorpholinium chloride, 2-chloro-4,6,-dimethoxy-1,3,5-triazine, cyanuric chloride, 1-ethyl-3-(3-dimethylaminopropyl)carbodiimide, benzotriazol-1-yloxytripyrrolidinophosphonium hexafluorophosphate, or 1-[bis(dimethylamino)methylene]-1H-1,2,3-triazolo[4,5-b]pyridinium 3-oxide hexafluorophosphate.

In some embodiments, the coupling agent is a carbonyl diimidazole, such as N,N′-dicyclohexylcarbodiimide (DCC), N,N′-dicyclopentylcarbodiimide, N,N′-diisopropylcarbodiimide (DIC), 1-ethyl-3-(3-dimethylaminopropyl) carbodiimide (EDC), N-t-butyl-N-methylcarbodiimide (BMC), N-t-butyl-N-ethylcarbodiimide (BEC), or 1,3-bis(2,2-dimethyl-1,3-dioxolan-4-ylmethyl)carbodiimide (BDDC).

In some embodiments, the conditions further comprise a solvent. In some embodiments, the conditions further comprise a solvent at a temperature of about −20° C. to about 60° C. In some embodiments, the conditions further comprise a solvent at a temperature of about −10° C. to about 10° C. In some embodiments, the conditions further comprise a solvent at a temperature of about 10° C. to about 30° C.

In some embodiments, the solvent is a polar aprotic solvent, an ether, an ester, a halogenated solvent, or a combination thereof, or a combination thereof with water. In some embodiments, the solvent is acetonitrile, N-methyl-2-pyrrolidone, dimethylformamide, dimethyl sulfoxide, tetrahydrofuran, 2-methytetrahydrofuran, ethyl acetate, isopropyl acetate, dichloromethane, or a combination thereof, or a combination thereof with water.

In some embodiments, the solvent is a polar aprotic solvent. In some embodiments, the polar aprotic solvent is N-methyl-2-pyrrolidone, dimethylformamide, dimethyl sulfoxide, or a combination thereof, or a combination thereof with water.

In some embodiments, the solvent is an ether. In some embodiments, the ether is tetrahydrofuran, 2-methytetrahydrofuran, or a combination thereof, or a combination thereof with water.

In some embodiments, the solvent is an ester. In some embodiments, the ester is ethyl acetate, isopropyl acetate, or a combination thereof, or a combination thereof with water.

In some embodiments, the solvent is a halogenated solvent. In some embodiments, the halogenated solvent is dichloromethane or a combination thereof, or a combination thereof with water.

In some embodiments, the solvent is acetonitrile.

In some embodiments, the conditions comprise N-methylimidazole and N,N,N′,N′-tetramethylchloroformamidinium hexafluorophosphate.

In some embodiments, the conditions further comprise acetonitrile at a temperature of about −10° C. to about 10° C.

In some embodiments, the conditions further comprise acetonitrile at a temperature of about 0° C.

In some embodiments, the conditions comprise N-methylimidazole and N,N,N′,N′-tetramethylchloroformamidinium hexafluorophosphate and acetonitrile solvent at a temperature of about 0° C.

In some embodiments, the conditions comprise N,N-diisopropylethylamine and diphenylphosphinic chloride. In some embodiments, the conditions further comprise acetonitrile at a temperature of about 10° C. to about 30° C. In some embodiments, the conditions further comprise acetonitrile at a temperature of about 20° C.

In some embodiments, the conditions comprise N,N-diisopropylethylamine, diphenylphosphinic chloride, and acetonitrile at a temperature of about 20° C.

In some embodiments, the process further comprises contacting Compound I with citric acid under conditions suitable to provide Compound I citric acid salt:

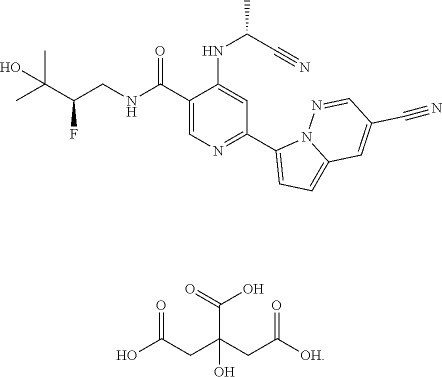 [SMILES: C[C@H](C#N)Nc1cc(-c2ccc3cc(C#N)cnn23)ncc1C(=O)NC[C@@H](F)C(C)(C)O ; O=C(O)CC(O)(CC(=O)O)C(=O)O]

In some embodiments, the conditions comprise a solvent. In some embodiments, the conditions comprise a solvent at a temperature of about 0° C. to about 90° C. In some embodiments, the conditions comprise a solvent at a temperature of about 45° C. to about 55° C. In some embodiments, the conditions comprise a solvent at a temperature of about 50° C.

In some embodiments, the solvent is an alcohol, an ether, an ester, a ketone, a nitrile, or a polar aprotic solvent. In some embodiments the solvent is ethanol, methanol, 1-propanol, 2-propanol, tetrahydrofuran, 2-methyltetrahydrofuran, tert-butyl methyl ether, ethyl acetate, isopropyl acetate, acetone, 2-butanone, 4-methyl-2-pentanone, acetonitrile, N,N-dimethylformamide, N,N-dimethylacetamide, or N-methyl-2-pyrrolidone.

In some embodiments, the solvent is an alcohol. In some embodiments, the alcohol is ethanol, methanol, 1-propanol, or 2-propanol.

In some embodiments, the solvent is an ether. In some embodiments, the ether is tetrahydrofuran, 2-methyltetrahydrofuran, or tert-butyl methyl ether.

In some embodiments, the solvent is an ester. In some embodiments, the ester is ethyl acetate or isopropyl acetate.

In some embodiments, the solvent is a ketone. In some embodiments, the ketone is acetone, 2-butanone, or 4-methyl-2-pentanone.

In some embodiments, the solvent is a nitrile. In some embodiments, the nitrile is acetonitrile.

In some embodiments, the solvent is a polar aprotic solvent. In some embodiments, the polar aprotic solvent is N,N-dimethylformamide, N,N-dimethylacetamide, or N-methyl-2-pyrrolidone.

In some embodiments, the solvent is ethanol.

In some embodiments, the conditions comprise ethanol at a temperature of about 45° C. to about 55° C. In some embodiments, the conditions comprise ethanol at a temperature of about 50° C.

In some embodiments, the conditions comprise contacting Compound I with about 1.5 equivalents of citric acid.

In some embodiments, the conditions comprise ethanol at a temperature of about 50° C. and contacting Compound I with about 1.5 equivalents of citric acid.

Also provided is a process for preparing Compound II, or a salt thereof:

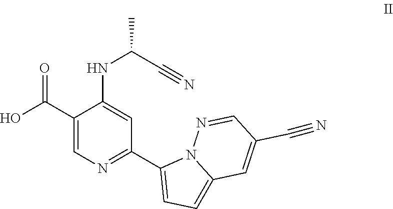 [SMILES: C[C@H](C#N)Nc1cc(-c2ccc3cc(C#N)cnn23)ncc1C(=O)O]

comprising contacting a compound of Formula IV, or a salt thereof;

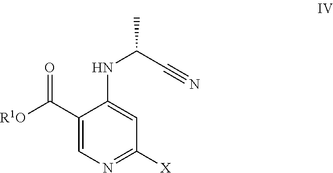

with a compound of Formula V, or a salt thereof:

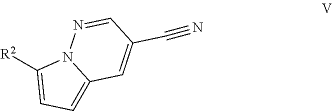

under conditions suitable to provide Compound II; wherein:
X is halo;
R¹ is a protecting group;
R² is hydrogen or —B(OR³)₂; and
each R³ is independently hydrogen or C₁₋₆ alkyl, or two R³ cyclize to form a cyclic boronate ester.

In some embodiments, R¹ is a carboxy protecting group. In certain embodiments, the carboxy protecting group is C₁₋₆ alkyl optionally substituted with C₁₋₆ alkoxy, cyano, —Si(C₁₋₆ alkyl)₃, or aryl, wherein the aryl is optionally substituted with one or more substituents independently selected from halo, nitro, C₁₋₆ alkyl, and C₁₋₆ alkoxy. In certain embodiments, R¹ is methyl, ethyl, n-propyl, isopropyl, n-butyl, isobutyl, tert-butyl, methoxymethyl, t-butoxymethyl, benzyl, p-methoxybenzyl, or p-nitrobenzyl. In some embodiments, R¹ is methyl, ethyl, n-propyl, iso-propyl, n-butyl, isobutyl, sec-butyl, tert-butyl, or benzyl. In some embodiments, R¹ is methyl, ethyl, tert-butyl, or benzyl. In some embodiments, R¹ is C₁₋₆ alkyl. In some embodiments, R¹ is methyl, ethyl, or tert-butyl. In some embodiments, R¹ is tert-butyl.

In some embodiments, R² is —B(OR³)₂. In some embodiments, two R³ cyclize to form a cyclic boronate ester. In some embodiments, R² is

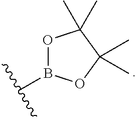 [SMILES: *B1OC(C)(C)C(C)(C)O1]

In some embodiments, R² is hydrogen.

In some embodiments, the conditions comprise a catalyst.

In some embodiments, the catalyst is a palladium(II) salt, a palladium(0) salt, copper(II) trifluoromethanesulfonate, or a combination thereof. In some embodiments, the catalyst is palladium(II) 2,4-pentanedionate, allylpalladium(II) chloride dimer, bis(acetonitrile)dichloropalladium(II), palladium(II) trifluoroacetate, palladium(II) chloride, tetrakis(triphenylphosphine)palladium(0), tris(dibenzylideneacetone)dipalladium(0), copper(II) trifluoromethanesulfonate, or a combination thereof. In some embodiments, the catalyst is a palladium(II) or palladium(0) salt.

In some embodiments, the catalyst is a palladium(II) salt. In some embodiments, the palladium(II) salt is palladium(II) 2,4-pentanedionate, allylpalladium(II) chloride dimer, bis(acetonitrile)dichloropalladium(II), palladium(II) trifluoroacetate, or palladium(II) chloride.

In some embodiments, the catalyst is a palladium(0) salt. In some embodiments, the palladium(0) salt is tetrakis(triphenylphosphine)palladium(0) or tris(dibenzylideneacetone)dipalladium(0).

In some embodiments, the catalyst is copper(II) trifluoromethanesulfonate. In some embodiments, the catalyst is a combination of copper(II) trifluoromethanesulfonate and palladium(II) and palladium(0) salts.

In some embodiments, the catalyst is palladium(II) acetate.

In some embodiments, the conditions comprise a catalyst and a ligand.

In some embodiments, the ligand is a dialkylbiaryl phosphine ligand, a bidentate phosphine ligand, or a monodentate phosphine ligand. In some embodiments, the ligand is 2-dicyclohexylphosphino-2′,6′-diisopropoxy biphenyl (RuPhos), 2-di-tert-butylphosphino-2′,4′,6′-triisopropylbiphenyl (t-BuXPhos), 2-dicyclohexylphosphino-2′,6′-dimethoxybiphenyl (SPhos), 2-(dicyclohexylphosphino) 3,6-dimethoxy-2′,4′,6′-triisopropyl-1,1′-biphenyl (BrettPhos), 2-dicyclohexylphosphino-2′,4′,6′-triisopropylbiphenyl (XPhos), 4,5-bis(diphenylphosphino)-9,9-dimethylxanthene (XantPhos), 1,1′-bis(di-tert-butylphosphino)ferrocene (dtbpf), 1,1′-ferrocenediyl-bis (diphenylphosphine) (dppf), triphenylphosphine, tri-tert-butyl phosphine, or tricyclohexyl phosphine. In some embodiments, the ligand is 2-dicyclohexylphosphino-2′,4′,6′-triisopropylbiphenyl (XPhos), 2-dicyclohexylphosphino-2′,6′-diisopropoxy biphenyl (RuPhos), a dialkylbiaryl phosphine ligand, a monodentate phosphine ligand, or a bidentate phosphine ligand.

In some embodiments, the ligand is a dialkylbiaryl phosphine ligand. In some embodiments, the dialkylbiaryl phosphine ligand is 2-dicyclohexylphosphino-2′,6′-diisopropoxybiphenyl (RuPhos), 2-di-tert-butylphosphino-2′,4′,6′-triisopropylbiphenyl (t-BuXPhos), 2-dicyclohexylphosphino-2′,6′-dimethoxy biphenyl (SPhos), 2-(dicyclohexylphosphino) 3,6-dimethoxy-2′,4′,6′-triisopropyl-1,1′-biphenyl (BrettPhos), or 2-dicyclohexylphosphino-2′,4′,6′-triisopropylbiphenyl (XPhos)).

In some embodiments, the ligand is a bidentate phosphine ligand. In some embodiments, the bidentate phosphine ligand is 4,5-bis(diphenylphosphino)-9,9-dimethylxanthene (XantPhos), 1,1′-bis (di-tert-butylphosphino)ferrocene (dtbpf), or 1,1′-ferrocenediyl-bis (diphenylphosphine) (dppf).

In some embodiments, the ligand is a monodentate phosphine ligand. In some embodiments, the monodentate phosphine ligand is triphenylphosphine, tri-tert-butyl phosphine, or tricyclohexyl phosphine.

In some embodiments, the ligand is 2-dicyclohexylphosphino-2′,6′-diisopropoxybiphenyl (RuPhos).

In some embodiments, the conditions comprise a catalyst, a ligand, and a base.

In some embodiments, the base is an inorganic base, an alkoxide base, a tertiary amine, or a nitrogen-containing heteroaryl base.

In some embodiments, the base is an amine, an inorganic base, an alkoxide base, an alkyl Grignard reagent, an alkyllithium, or a diorganylamide base. In some embodiments, the base is N,N-diisopropylamine, triethylamine, 1,8-diazabicyclo[5.4.0]undec-7-ene, N-methylimidazole, sodium hydroxide, lithium hydroxide, potassium hydroxide, calcium hydroxide, magnesium hydroxide, tetramethylammonium hydroxide, tetrabutylammonium hydroxide, sodium carbonate, lithium carbonate, potassium carbonate, calcium carbonate, magnesium carbonate, tetramethylammonium carbonate, tetrabutylammonium carbonate, sodium phosphate mono/di/tribasic, lithium phosphate mono/di/tribasic, potassium phosphate mono/di/tribasic, calcium phosphate mono/di/tribasic, magnesium phosphate mono/di/tribasic, tetramethylammonium phosphate mono/di/tribasic, tetrabutylammonium phosphate mono/di/tribasic, sodium methoxide, lithium methoxide, potassium methoxide, magnesium methoxide, calcium methoxide, sodium ethoxide, lithium ethoxide, potassium ethoxide, magnesium ethoxide, calcium ethoxide, sodium tert-butoxide, lithium tert-butoxide, potassium tert-butoxide, magnesium tert-butoxide, calcium tert-butoxide, methylmagnesium bromide, methylmagnesium chloride, isopropylmagnesium chloride, methyllithium, n-butyllithium, sodium diethylamide, potassium diethylamide, lithium diethylamide, sodium diisopropylamide, potassium diisopropylamide, or lithium diisopropylamide.

In some embodiments, the base is an amine. In some embodiments, the amine is N,N-diisopropylamine, triethylamine, 1,8-diazabicyclo[5.4.0]undec-7-ene, or N-methylimidazole.

In some embodiments, the base is an inorganic base. In some embodiments, the inorganic base is sodium, lithium, potassium, calcium, magnesium, tetramethylammonium, or tetrabutylammonium salts of hydroxide, carbonate, phosphate tribasic, phosphate dibasic, or phosphate monobasic.

In some embodiments, the base is an alkoxide base. In some embodiments, the alkoxide base is sodium, lithium, potassium, magnesium, or calcium salts of methoxide, ethoxide, or tert-butoxide.

In some embodiments, the base is an alkyl Grignard reagent. In some embodiments, the alkyl Grignard reagent is methylmagnesium bromide, methylmagnesium chloride, or isopropylmagnesium chloride.

In some embodiments, the base is an alkyllithium. In some embodiments, the alkyllithium is methyllithium or n-butyllithium.

In some embodiments, the base a diorganylamide. In some embodiments, the diorganylamide base is sodium, potassium, or lithium salts of diethylamide or diisopropylamide.

In some embodiments, the base is potassium carbonate.

In some embodiments, the conditions further comprise a solvent. In some embodiments, the conditions further comprise a solvent at a temperature of about 45° C. to about 110° C. In some embodiments, the conditions further comprise a solvent at a temperature of about 65° C. to about 85° C.

In some embodiments, the solvent is an alcohol, an ether, a hydrocarbon, an ester, an aqueous surfactant, or a combination thereof, or a combination thereof with water. In some embodiments, the solvent is methanol, ethanol, 2-propanol, 2-methyltetrahydrofuran, tert-butyl methyl ether, n-heptane, toluene, ethyl acetate, isopropyl acetate, Coolade (CAS No. 2306441-11-0), TPGS-750-M (DL-a-Tocopherol methoxypolyethylene glycol succinate), hydroxypropyl methylcellulose, or a combination thereof, or a combination thereof with water.

In some embodiments, the solvent is an alcohol. In some embodiments, the alcohol is methanol, ethanol. 2-propanol, or a combination thereof, or a combination thereof with water.

In some embodiments, the solvent is an ether. In some embodiments, the ether is 2-methyltetrahydrofuran, tert-butyl methyl ether, or a combination thereof, or a combination thereof with water.

In some embodiments, the solvent is a hydrocarbon. In some embodiments, the hydrocarbon is n-heptane, toluene, or a combination thereof, or a combination thereof with water.

In some embodiments, the solvent is an ester. In some embodiments, the ester is ethyl acetate, isopropyl acetate, or a combination thereof, or a combination thereof with water.

In some embodiments, the solvent is an aqueous surfactant. In some embodiments, the aqueous surfactant is Coolade (CAS No. 2306441-11-0), TPGS-750-M (DL-a-tocopherol methoxypolyethylene glycol succinate), hydroxypropyl methylcellulose, or a combination thereof, or a combination thereof with water.

In some embodiments, the solvent is tetrahydrofuran and water.

In some embodiments, the conditions comprise potassium carbonate, palladium(II) acetate, and 2-dicyclohexylphosphino-2′,6′-diisopropoxybiphenyl (RuPhos). In some embodiments, the conditions further comprise tetrahydrofuran and water at a temperature of about 65° C. to about 85° C. In some embodiments, the conditions further comprise tetrahydrofuran and water at a temperature of about 75° C.

In some embodiments, the conditions comprise potassium carbonate, palladium(II) acetate, 2-dicyclohexylphosphino-2′,6′-diisopropoxybiphenyl (RuPhos), tetrahydrofuran, and water at a temperature of about 75° C.

Also provided is a process for preparing Compound II, or a salt thereof:

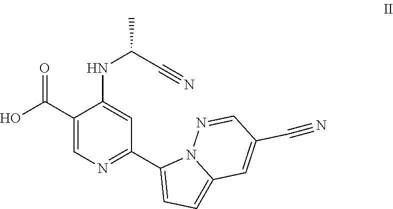 [SMILES: C[C@H](C#N)Nc1cc(-c2ccc3cc(C#N)cnn23)ncc1C(=O)O]

comprising contacting a compound of Formula IV, or a salt thereof;

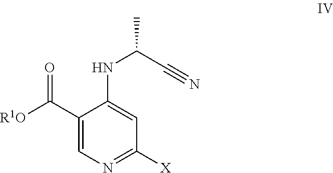

with a compound of Formula V, or a salt thereof:

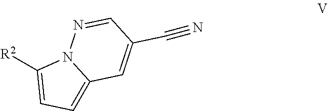

under conditions suitable to provide Compound I; wherein:
X is halo;
R¹ is a protecting group; and
R² is hydrogen.

In some embodiments, R¹ is a carboxy protecting group. In certain embodiments, the carboxy protecting group is C₁₋₆ alkyl optionally substituted with C₁₋₆ alkoxy, cyano, —Si(C₁₋₆ alkyl)₃, or aryl, wherein the aryl is optionally substituted with one or more substituents independently selected from halo, nitro, C₁₋₆ alkyl, and C₁₋₆ alkoxy. In certain embodiments, R¹ is methyl, ethyl, n-propyl, isopropyl, n-butyl, isobutyl, tert-butyl, methoxymethyl, t-butoxymethyl, benzyl, p-methoxybenzyl, or p-nitrobenzyl. In some embodiments, R¹ is methyl, ethyl, n-propyl, isopropyl, n-butyl, isobutyl, sec-butyl, tert-butyl, or benzyl. In some embodiments, R¹ is methyl, ethyl, tert-butyl, or benzyl.

In some embodiments, R¹ is C₁₋₆ alkyl. In some embodiments, R¹ is methyl, ethyl, or tert-butyl. In some embodiments, R¹ is tert-butyl.

In some embodiments, the conditions comprise a catalyst, a ligand, and an acid.

In some embodiments, the catalyst is a palladium catalyst or a palladium precatalyst. In some embodiments, the catalyst is palladium acetate, allylpalladium chloride dimer, cinnamylpalladium chloride dimer, (η3-crotyl) palladium chloride tri-tert-butylphosphine, or 2-dicyclohexylphosphino-2′,4′,6′-triisopropyl-1,1′-biphenyl)[2-(2′-amino-1,1′-biphenyl)]palladium(II) methanesulfonate.

In some embodiments, the catalyst is an allylpalladium chloride dimer.

In some embodiments, the ligand is a monodentate phosphine or a bidentate phosphine. In some embodiments, the ligand is tri-tert-butylphosphine, 2-dicyclohexylphosphino-2′,4′,6′-triisopropylbiphenyl (XPhos), 2-dicyclohexylphosphino-2′,6′-dimethoxybiphenyl (SPhos), triphenylphosphine, 4,5-bis (diphenylphosphino)-9,9-dimethylxanthene (XantPhos), or 2,2′-bis(diphenylphosphino)-1,1′-binaphthyl (BINAP).

In some embodiments, the ligand is a monodentate phosphine. In some embodiments, the monodentate phosphine is tri-tert-butylphosphine, 2-dicyclohexylphosphino-2′,4′,6′-triisopropylbiphenyl (XPhos), 2-dicyclohexylphosphino-2′,6′-dimethoxybiphenyl (SPhos), or triphenylphosphine.

In some embodiments, the ligand is a bidentate phosphine. In some embodiments, the bidentate phosphine is 4,5-bis(diphenylphosphino)-9,9-dimethylxanthene (XantPhos) or 2,2′-bis (diphenylphosphino)-1,1′-binaphthyl (BINAP).

In some embodiments, the ligand is tri-tert-butylphosphonium tetrafluoroborate.

In some embodiments, the acid is a carboxylic acid or a sulfonic acid. In some embodiments, the acid is acetic acid, isobutyric acid, pivalic acid, 1-adamantane carboxylic acid, benzoic acid, methanesulfonic acid, benzenesulfonic acid, or toluenesulfonic acid.

In some embodiments, the acid is a carboxylic acid. In some embodiments, the carboxylic acid is acetic acid, 2,2-dimethylbutyric acid, isobutyric acid, pivalic acid, 1-adamantane carboxylic acid, or benzoic acid.

In some embodiments, the acid is a sulfonic acid. In some embodiments, the sulfonic acid is methanesulfonic acid, benzenesulfonic acid, or toluenesulfonic acid.

In some embodiments, the acid is 2,2-dimethylbutyric acid.

In some embodiments, the conditions further comprise a base. In some embodiments, the base is an amine, an inorganic base, an alkoxide base, an alkyl Grignard reagent, an alkyllithium, or a diorganylamide base. In some embodiments, the base is N,N-diisopropylamine, triethylamine, 1,8-diazabicyclo[5.4.0]undec-7-ene, N-methylimidazole, sodium hydroxide, lithium hydroxide, potassium hydroxide, calcium hydroxide, magnesium hydroxide, tetramethylammonium hydroxide, tetrabutylammonium hydroxide, sodium carbonate, lithium carbonate, potassium carbonate, calcium carbonate, magnesium carbonate, tetramethylammonium carbonate, tetrabutylammonium carbonate, sodium phosphate mono/di/tribasic, lithium phosphate mono/di/tribasic, potassium phosphate mono/di/tribasic, calcium phosphate mono/di/tribasic, magnesium phosphate mono/di/tribasic, tetramethylammonium phosphate mono/di/tribasic, tetrabutylammonium phosphate mono/di/tribasic, sodium methoxide, lithium methoxide, potassium methoxide, magnesium methoxide, calcium methoxide, sodium ethoxide, lithium ethoxide, potassium ethoxide, magnesium ethoxide, calcium ethoxide, sodium tert-butoxide, lithium tert-butoxide, potassium tert-butoxide, magnesium tert-butoxide, calcium tert-butoxide, methylmagnesium bromide, methylmagnesium chloride, isopropylmagnesium chloride, methyllithium, n-butyllithium, sodium diethylamide, potassium diethylamide, lithium diethylamide, sodium diisopropylamide, potassium diisopropylamide, or lithium diisopropylamide.

In some embodiments, the base is potassium bicarbonate.

In some embodiments, the conditions further comprise a solvent. In some embodiments, the conditions further comprise a solvent at a temperature of about 60° C. to about 140° C. In some embodiments, the temperature is about 70° C. to about 110° C. In some embodiments, the temperature is about 90° C.

In some embodiments, the solvent is an alcohol, an ether, a hydrocarbon, an ester, or an aqueous surfactant. In some embodiments, the solvent is methanol, ethanol, 2-propanol, 2-methyltetrahydrofuran, tert-butyl methyl ether, n-heptane, toluene, ethyl acetate, isopropyl acetate, Coolade (CAS No. 2306441-11-0), TPGS-750-M (DL-a-tocopherol methoxypolyethylene glycol succinate), or hydroxypropyl methylcellulose.

In some embodiments, the solvent is an alcohol. In some embodiments, the alcohol is 2-butanol, methanol, ethanol or 2-propanol.

In some embodiments, the solvent is an ether. In some embodiments, the ether is 2-methyltetrahydrofuran or tert-butyl methyl ether.

In some embodiments, the solvent is a hydrocarbon. In some embodiments, the hydrocarbon is n-heptane or toluene.

In some embodiments, the solvent is an ester. In some embodiments, the ester is ethyl acetate or isopropyl acetate.

In some embodiments, the solvent is an aqueous surfactant. In some embodiments, the aqueous surfactant is Coolade (CAS No. 2306441-11-0), TPGS-750-M (DL-a-tocopherol methoxypolyethylene glycol succinate), or hydroxypropyl methylcellulose.

In some embodiments, the solvent is 2-butanol.

In some embodiments, the conditions comprise an allylpalladium chloride dimer, tri-tert-butylphosphonium tetrafluoroborate, and 2,2-dimethylbutyric acid. In some embodiments, the conditions further comprise potassium bicarbonate. In some embodiments, the conditions further comprise 2-butanol at a temperature of about 70° C. to about 110° C. In some embodiments, the conditions further comprise 2-butanol at a temperature of about 90° C.

In some embodiments, the conditions comprise an allylpalladium chloride dimer, tri-tert-butylphosphonium tetrafluoroborate, 2,2-dimethylbutyric acid, potassium bicarbonate, and 2-butanol at a temperature of about 90° C.

In some embodiments, the conditions further comprise a deprotection step.

In some embodiments, the deprotection step comprises converting a compound of Formula IIA to Compound II:

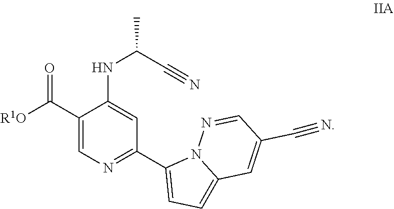

In some embodiments, R¹ is a carboxy protecting group. In certain embodiments, the carboxy protecting group is C₁₋₆ alkyl optionally substituted with C₁₋₆ alkoxy, cyano, —Si(C₁₋₆ alkyl)₃, or aryl, wherein the aryl is optionally substituted with one or more substituents independently selected from halo, nitro, C₁₋₆ alkyl, and C₁₋₆ alkoxy. In certain embodiments, R¹ is methyl, ethyl, n-propyl, isopropyl, n-butyl, isobutyl, tert-butyl, methoxymethyl, t-butoxymethyl, benzyl, p-methoxybenzyl, or p-nitrobenzyl.

In some embodiments. R¹ is methyl, ethyl, n-propyl, isopropyl, n-butyl, isobutyl, sec-butyl, tert-butyl, or benzyl. In some embodiments, R¹ is methyl, ethyl, tert-butyl, or benzyl.

In some embodiments, R¹ is C₁₋₆ alkyl. In some embodiments, R¹ is methyl, ethyl, or tert-butyl. In some embodiments, R¹ is tert-butyl.

In some embodiments, the deprotection step comprises a reagent and an optional additive. In some embodiments, the deprotection step comprises methanesulfonic acid.

In some embodiments, the reagent is an inorganic acid, organic acid, an acid chloride, or a Lewis acid. In some embodiments, the reagent is hydrobromic acid, hydroiodic acid, sulfuric acid, phosphoric acid, formic acid, acetic acid, trifluoroacetic acid, citric acid, oxalic acid, benzenesulfonic acid, toluenesulfonic acid, camphorsulfonic acid, acetyl chloride, aluminum chloride, aluminum bromide, lithium chloride, magnesium chloride, boron trichloride, boron tribromide, samarium iodide, or lanthanum triflate.

In some embodiments, the reagent is an inorganic acid. In some embodiments, the inorganic acid is hydrobromic acid, hydroiodic acid, sulfuric acid, or phosphoric acid.

In some embodiments, the reagent is an organic acid. In some embodiments, the organic acid is formic acid, acetic acid, trifluoroacetic acid, citric acid, oxalic acid, benzenesulfonic acid, toluenesulfonic acid, or camphorsulfonic acid.

In some embodiments, the reagent is an acid chloride. In some embodiments, the acid chloride is an acyl halide (e.g., acetyl chloride, propionyl chloride, butyryl chloride, benzoyl chloride, chloroacetyl chloride, trifluoroacetyl chloride, or p-toluenesulfonyl chloride), thionyl chloride, or phosphorus pentachloride, and the like. In some embodiments, the acyl halide is acetyl chloride.

In some embodiments, the reagent is a Lewis acid. In some embodiments, the Lewis acid is aluminum chloride, aluminum bromide, lithium chloride, magnesium chloride, boron trichloride, boron tribromide, samarium iodide, or lanthanum triflate.

In some embodiments, the reagent is methanesulfonic acid.

In some embodiments, the optional additive is a trialkylsilane. In some embodiments, the additive is trimethylsilane or triethylsilane. In some embodiments, the optional additive is absent.

In some embodiments, the solvent is an ether, a hydrocarbon, a nitrile, an ester, a halogenated solvents, a polar aprotic solvent, an alcohol, a ketone, or a carboxylic acid. In some embodiments, the solvent is tetrahydrofuran, 2-methyltetrahydrofuran, methyl tert-butyl ether, cyclopentyl methyl ether, 1,4-dioxane, n-heptane, toluene, butyronitrile, ethyl acetate, isopropyl acetate, dichloromethane, 1,2-dichloroethane, N,N-dimethylformamide, N-methyl-2-pyrrolidone, ethanol, isopropanol, 1-butanol, 2-butanol, acetone, formic acid, or acetic acid.

In some embodiments, the deprotection step further comprises a solvent. In some embodiments, the deprotection step further comprises a solvent at a temperature of about 0° C. to about 120° C. In some embodiments, the deprotection step further comprises a solvent at a temperature of about 55° C. to about 75° C. In some embodiments, the deprotection step further comprises a solvent at a temperature of about 65° C.

In some embodiments, the solvent is an ether. In some embodiments, the ether is tetrahydrofuran, 2-methyltetrahydrofuran, methyl tert-butyl ether, cyclopentyl methyl ether, or 1,4-dioxane.

In some embodiments, the solvent is a hydrocarbon. In some embodiments, the hydrocarbon is n-heptane or toluene.

In some embodiments, the solvent is a nitrile. In some embodiments, the nitrile is butyronitrile or acetonitrile.

In some embodiments, the solvent is an ester. In some embodiments, the ester is ethyl acetate or isopropyl acetate.

In some embodiments, the solvent is a halogenated solvent. In some embodiments, the halogenated solvent is dichloromethane or 1,2-dichloroethane.

In some embodiments, the solvent is a polar aprotic solvent. In some embodiments, the polar aprotic solvent is N,N-dimethylformamide or N-methyl-2-pyrrolidone.

In some embodiments, the solvent is an alcohol. In some embodiments, the alcohol is methanol, ethanol, isopropanol, 1-butanol, or 2-butanol.

In some embodiments, the solvent is a ketone. In some embodiments, the ketone is acetone.

In some embodiments, the solvent is a carboxylic acid. In some embodiments, the carboxylic acid is formic acid or acetic acid.

In some embodiments, the solvent is acetonitrile.

In some embodiments, the deprotection step comprises methanesulfonic acid. In some embodiments, the deprotection step further comprises acetonitrile at a temperature of about 55° C. to about 75° C. In some embodiments, the deprotection step further comprises acetonitrile at a temperature of about 65° C.

In some embodiments, the deprotection step comprises methanesulfonic acid and acetonitrile at a temperature of about 65° C.

In some embodiments, the process provides Compound II methanesulfonic acid salt:

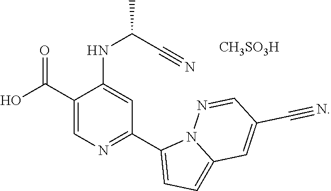 [SMILES: C[C@H](C#N)Nc1cc(-c2ccc3cc(C#N)cnn23)ncc1C(=O)O ; "CH3SO3H"]

Also provided herein is a process for preparing Compound I, or a salt thereof:

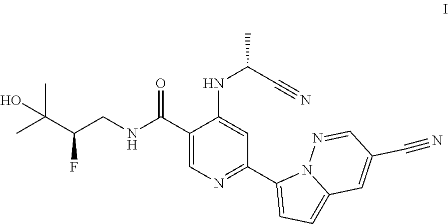 [SMILES: C[C@H](C#N)Nc1cc(-c2ccc3cc(C#N)cnn23)ncc1C(=O)NC[C@@H](F)C(C)(C)O]

comprising contacting compound of Formula VI, or a salt thereof:

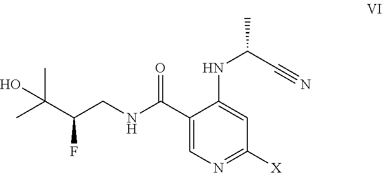

with a compound of Formula V, or a salt thereof:

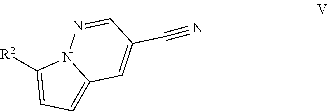

under conditions suitable to provide Compound I; provided the conditions do not comprise microwave radiation; wherein:
X is halo;
R² is hydrogen or —B(OR³)₂; and
each R³ is independently hydrogen or C₁₋₆ alkyl, or two R³ cyclize to form a cyclic boronate ester.

In some embodiments, R² is —B(OR³)₂.

In some embodiments, the compound of Formula V is represented by a compound of Formula VA:

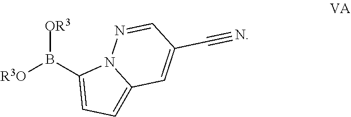

In some embodiments, two R³ cyclize to form a cyclic boronate ester. In some embodiments, R² is

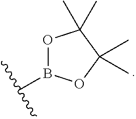 [SMILES: *B1OC(C)(C)C(C)(C)O1]

In some embodiments, the conditions comprise a catalyst, a ligand, and a base.

In some embodiments, the catalyst is a palladium(II) or a palladium(0) salt. In some embodiments, the catalyst is palladium(II) acetate, palladium(II) 2,4-pentanedionate (Pd(acac)₂), allylpalladium(II) chloride dimer ([Pd(allyl)Cl₂]), bis(acetonitrile)dichloropalladium(II) (Pd(MeCN)₂Cl₂), tris(dibenzylideneacetone)dipalladium(0) (Pd(dba)₃), palladium(II) trifluoroacetate (Pd(TFA)₂), tetrakis(triphenylphosphine)palladium(0) (Pd(PPh₃)₄), or palladium(II) chloride (PdCl₂).

In some embodiments, the catalyst is a palladium(II) salt. In some embodiments, the palladium(II) salt is palladium(II) acetate, palladium(II) 2,4-pentanedionate (Pd(acac)₂), allylpalladium(II) chloride dimer ([Pd(allyl)Cl₂]), bis(acetonitrile)dichloropalladium(II) (Pd(MeCN)₂Cl₂), palladium(II) trifluoroacetate (Pd(TFA)₂), palladium(II) chloride (PdCl₂).

In some embodiments, the catalyst is a palladium(0) salt. In some embodiments, the palladium(0) salt is tris(dibenzylideneacetone)dipalladium(0) (Pd(dba)₃) or tetrakis(triphenylphosphine)palladium(0) (Pd(PPh₃)₄).

In some embodiments, the catalyst is palladium(II) acetate.

In some embodiments, the ligand is a dialkylbiaryl phosphine ligand, a monodentate phosphine ligand, or a bidentate phosphine ligand. In some embodiments, the ligand is 2-di-tert-butylphosphino-2′,4′,6′-triisopropylbiphenyl (t-BuXPhos), 2-dicyclohexylphosphino-2′,6′-dimethoxybiphenyl (SPhos), 2-(dicyclohexylphosphino)3,6-dimethoxy-2′,4′,6′-triisopropyl-1,1′-biphenyl (BrettPhos), 2-dicyclohexylphosphino-2′,6′-diisopropoxybiphenyl (RuPhos), triphenylphosphine, tri-tert-butyl phosphine, tricyclohexyl phosphine, 4,5-bis(diphenylphosphino)-9,9-dimethylxanthene (XantPhos), 1,1′-bis(di-tert-butylphosphino)ferrocene (dtbpf), or 1,1′-ferrocenediyl-bis(diphenylphosphine) (dppf).

In some embodiments, the ligand is a dialkylbiaryl phosphine ligand. In some embodiments, the dialkylbiaryl phosphine ligand is 2-di-tert-butylphosphino-2′,4′,6′-triisopropylbiphenyl (t-BuXPhos), 2-dicyclohexylphosphino-2′,6′-dimethoxybiphenyl (SPhos), 2-(dicyclohexylphosphino)3,6-dimethoxy-2′,4′,6′-triisopropyl-1,1′-biphenyl (BrettPhos), or 2-dicyclohexylphosphino-2′,6′-diisopropoxybiphenyl (RuPhos).

In some embodiments, the ligand is a monodentate phosphine ligand. In some embodiments, the monodentate phosphine ligand is triphenylphosphine, tri-tert-butyl phosphine, or tricyclohexyl phosphine.

In some embodiments, the ligand is a bidentate phosphine ligand. In some embodiments, the bidentate phosphine ligand is 4,5-bis(diphenylphosphino)-9,9-dimethylxanthene (XantPhos), 1,1′-bis (di-tert-butylphosphino)ferrocene (dtbpf), or 1,1′-ferrocenediyl-bis (diphenylphosphine) (dppf).

In some embodiments, the ligand is 2-dicyclohexylphosphino-2′,4′,6′-triisopropylbiphenyl (XPhos).

In some embodiments, the base is an inorganic base, an alkoxide base, or an organic base, such as, but not limited to, an amine base, e.g., a tertiary amine, or a nitrogen-containing heterocyclic or nitrogen-containing heteroaryl base. In some embodiments, the base is sodium hydroxide, lithium hydroxide, potassium hydroxide, calcium hydroxide, magnesium hydroxide, tetramethylammonium hydroxide, tetrabutylammonium hydroxide, sodium carbonate, lithium carbonate, potassium carbonate, calcium carbonate, magnesium carbonate, tetramethylammonium carbonate, tetrabutylammonium carbonate, sodium phosphate mono/di/tribasic, lithium phosphate mono/di/tribasic, potassium phosphate mono/di/tribasic, calcium phosphate mono/di/tribasic, magnesium phosphate mono/di/tribasic, tetramethylammonium phosphate mono/di/tribasic, tetrabutylammonium phosphate mono/di/tribasic, sodium methoxide, lithium methoxide, potassium methoxide, magnesium methoxide, calcium methoxide, sodium ethoxide, lithium ethoxide, potassium ethoxide, magnesium ethoxide, calcium ethoxide, sodium tert-butoxide, lithium tert-butoxide, potassium tert-butoxide, N-methylmorpholine, triethylamine, N-methylmorpholine, triethylamine, pyridine, or N-methylimidazole.

In some embodiments, the base is an inorganic base. In some embodiments, the inorganic base is sodium hydroxide, lithium hydroxide, potassium hydroxide, calcium hydroxide, magnesium hydroxide, tetramethylammonium hydroxide, tetrabutylammonium hydroxide, sodium carbonate, lithium carbonate, potassium carbonate, calcium carbonate, magnesium carbonate, tetramethylammonium carbonate, tetrabutylammonium carbonate, sodium phosphate mono/di/tribasic, lithium phosphate mono/di/tribasic, potassium phosphate mono/di/tribasic, calcium phosphate mono/di/tribasic, magnesium phosphate mono/di/tribasic, tetramethylammonium phosphate mono/di/tribasic, or tetrabutylammonium phosphate mono/di/tribasic.

In some embodiments, the base is an alkoxide base. In some embodiments, the alkoxide base is sodium methoxide, lithium methoxide, potassium methoxide, magnesium methoxide, calcium methoxide, sodium ethoxide, lithium ethoxide, potassium ethoxide, magnesium ethoxide, calcium ethoxide, sodium tert-butoxide, lithium tert-butoxide, or potassium tert-butoxide.

In some embodiments, the base is an amine base. In some embodiments, the amine base is triethylamine, diisopropylethyl amine, tributyl amine, N-methylmorpholine, pyridine, 4-dimethylaminopyridine, or N-methylimidazole. In some embodiments, the base is a tertiary amine. In some embodiments, the tertiary amine is N-methylmorpholine or triethylamine.

In some embodiments, the base is a nitrogen-containing heterocyclic or nitrogen-containing heteroaryl base. In some embodiments, the nitrogen-containing heterocyclic or nitrogen-containing heteroaryl base is N-methylmorpholine, pyridine, or N-methylimidazole.

In some embodiments, the base is potassium phosphate tribasic.

In some embodiments, the conditions further comprise a solvent. In some embodiments, the conditions further comprise a solvent at a temperature of about 45° C. to about 110° C. In some embodiments, the conditions further comprise a solvent at a temperature of about 70° C. to about 80° C. In some embodiments, the conditions further comprise a solvent at a temperature of about 75° C.

In some embodiments, the solvent is an ether, a ketone, a polar aprotic solvent, a hydrocarbon, a nitrile, or a combination thereof, or a combination thereof with water. In some embodiments, the solvent is tetrahydrofuran, tert-butyl methyl ether, cyclopentyl methyl ether (CPME), acetone, 2-butanone, 4-methyl-2-pentanone, N,N-dimethylformamide, N,N-dimethylacetamide, N-methyl-2-pyrrolidone, toluene, n-heptane, acetonitrile, or a combination thereof, or a combination thereof with water.

In some embodiments, the solvent is an ether. In some embodiments, the ether is tetrahydrofuran, tert-butyl methyl ether, cyclopentyl methyl ether (CPME), or a combination thereof, or a combination thereof with water.

In some embodiments, the solvent is a ketone. In some embodiments, the ketone is acetone, 2-butanone, 4-methyl-2-pentanone, or a combination thereof, or a combination thereof with water.

In some embodiments, the solvent is a polar aprotic solvent. In some embodiments, the polar aprotic solvent is N,N-dimethylformamide, N,N-dimethylacetamide, N-methyl-2-pyrrolidone, or a combination thereof, or a combination thereof with water.

In some embodiments, the solvent is a hydrocarbon. In some embodiments, the hydrocarbon is toluene, n-heptane, or a combination thereof, or a combination thereof with water.

In some embodiments, the solvent is a nitrile. In some embodiments, the nitrile is acetonitrile or a combination thereof, or a combination thereof with water.

In some embodiments, the solvent is 2-methyltetrahydrofuran and water.

In some embodiments, the conditions comprise palladium(II) acetate, 2-dicyclohexylphosphino-2′,4′,6′-triisopropylbiphenyl (XPhos), and potassium phosphate tribasic. In some embodiments, the conditions further comprise 2-methyltetrahydrofuran and water at a temperature of from about 70° C. to about 80° C. In some embodiments, the conditions further comprise 2-methyltetrahydrofuran and water at a temperature of about 75° C.

In some embodiments, the conditions comprise palladium(II) acetate, 2-dicyclohexylphosphino-2′,4′,6′-triisopropylbiphenyl (XPhos), potassium phosphate tribasic, 2-methyltetrahydrofuran, and water at a temperature of about 75° C.

In some embodiments, the process further comprises contacting Compound I with citric acid to provide Compound I citric acid salt:

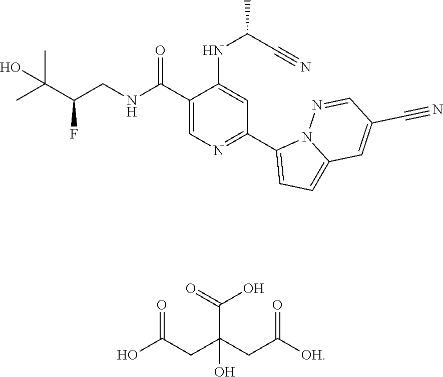 [SMILES: C[C@H](C#N)Nc1cc(-c2ccc3cc(C#N)cnn23)ncc1C(=O)NC[C@@H](F)C(C)(C)O ; O=C(O)CC(O)(CC(=O)O)C(=O)O]

In some embodiments, the conditions comprise a solvent. In some embodiments, the conditions comprise a solvent at a temperature of about 0° C. to about 90° C. In some embodiments, the conditions comprise a solvent at a temperature of about 45° C. to about 55° C. In some embodiments, the conditions comprise a solvent at a temperature of about 50° C.

In some embodiments, the solvent is an alcohol, an ether, an ester, a ketone, a nitrile, or a polar aprotic solvent. In some embodiments the solvent is ethanol, methanol, 1-propanol, 2-propanol, tetrahydrofuran, 2-methyltetrahydrofuran, tert-butyl methyl ether, ethyl acetate, isopropyl acetate, acetone, 2-butanone, 4-methyl-2-pentanone, acetonitrile, N,N-dimethylformamide, N,N-dimethylacetamide, or N-methyl-2-pyrrolidone.

In some embodiments, the solvent is an alcohol. In some embodiments, the alcohol is ethanol, methanol, 1-propanol, or 2-propanol.

In some embodiments, the solvent is an ether. In some embodiments, the ether is tetrahydrofuran, 2-methyltetrahydrofuran, or tert-butyl methyl ether.

In some embodiments, the solvent is an ester. In some embodiments, the ester is ethyl acetate or isopropyl acetate.

In some embodiments, the solvent is a ketone. In some embodiments, the ketone is acetone, 2-butanone, or 4-methyl-2-pentanone.

In some embodiments, the solvent is a nitrile. In some embodiments, the nitrile is acetonitrile.

In some embodiments, the solvent is a polar aprotic solvent. In some embodiments, the polar aprotic solvent is N,N-dimethylformamide, N,N-dimethylacetamide, or N-methyl-2-pyrrolidone.

In some embodiments, the solvent is ethanol.

In some embodiments, the conditions comprise ethanol at a temperature of about 45° C. to about 55° C. In some embodiments, the conditions comprise ethanol at a temperature of about 50° C.

In some embodiments, the process comprises contacting Compound I with about 1.5 equivalents of citric acid.

In some embodiments, the conditions comprise ethanol at a temperature of about 50° C. and contacting Compound I with about 1.5 equivalents of citric acid.

Also provided herein is a process for preparing a compound of Formula VI, or a salt thereof:

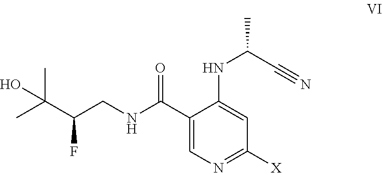

comprising contacting a compound of Formula VII, or a salt thereof:

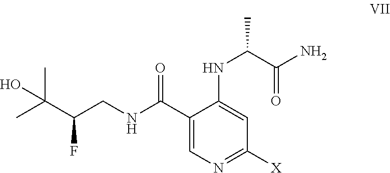

with trifluoroacetic anhydride in the absence of an organic base, under conditions suitable to provide a compound of Formula VIII:

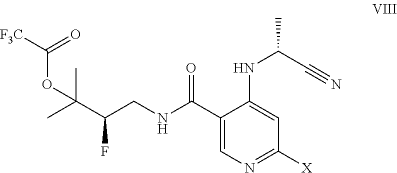

wherein X is halo; and hydrolyzing a compound of Formula VIII to provide a compound of Formula VI.

In some embodiments, the hydrolyzing comprises an inorganic base or an alkoxide base.

In some embodiments, the inorganic base is sodium hydroxide, lithium hydroxide, potassium hydroxide, calcium hydroxide, magnesium hydroxide, tetramethylammonium hydroxide, tetrabutylammonium hydroxide, sodium carbonate, lithium carbonate, potassium carbonate, calcium carbonate, magnesium carbonate, tetramethylammonium carbonate, tetrabutylammonium carbonate, sodium phosphate mono/di/tribasic, lithium phosphate mono/di/tribasic, potassium phosphate mono/di/tribasic, calcium phosphate mono/di/tribasic, magnesium phosphate mono/di/tribasic, tetramethylammonium phosphate mono/di/tribasic, or tetrabutylammonium phosphate mono/di/tribasic.

In some embodiments, the alkoxide base is sodium methoxide, lithium methoxide, potassium methoxide, magnesium methoxide, calcium methoxide, sodium ethoxide, lithium ethoxide, potassium ethoxide, magnesium ethoxide, calcium ethoxide, sodium tert-butoxide, lithium tert-butoxide, or potassium tert-butoxide.

In some embodiments, the base is potassium carbonate.

In some embodiments, the conditions comprise a solvent. In some embodiments, the conditions comprise a solvent at a temperature of about −20° C. to about 60° C. In some embodiments, the conditions comprise a solvent at a temperature of about −10° C. to about 10° C. In some embodiments, the conditions comprise a solvent at a temperature of about 0° C.

In some embodiments, the solvent is an ester, an ether, a ketone, a nitrile, a hydrocarbon, a halogenated solvent, a polar aprotic solvent, or a combination thereof, or a combination thereof with water. In some embodiments, the solvent is ethyl acetate, isopropyl acetate, 2-methyltetrahydrofuran, tert-butyl methyl ether, acetone, 2-butanone, 4-methyl-2-pentanone, toluene, n-heptane, dichloromethane, 1,2-dichloroethane, chlorobenzene, acetonitrile, N,N-dimethylformamide, N,N-dimethylacetamide, N-methyl-2-pyrrolidone, or a combination thereof, or a combination thereof with water.

In some embodiments, the solvent is an ester. In some embodiments, the ester is ethyl acetate, isopropyl acetate, or a combination thereof, or a combination thereof with water.

In some embodiments, the solvent is an ether. In some embodiments, the ether is tetrahydrofuran, 2-methyltetrahydrofuran, tert-butyl methyl ether, or a combination thereof with water.

In some embodiments, the solvent is a ketone. In some embodiments, the ketone is acetone, 2-butanone, 4-methyl-2-pentanone, or a combination thereof, or a combination thereof with water.

In some embodiments, the solvent is a nitrile. In some embodiments, the nitrile is acetonitrile or a combination thereof, or a combination thereof with water.

In some embodiments, the solvent is a hydrocarbon. In some embodiments, the hydrocarbon is toluene or n-heptane, or a combination thereof, or a combination thereof with water.

In some embodiments, the solvent is a halogenated solvent. In some embodiments, the halogenated solvent is dichloromethane, 1,2-dichloroethane, chlorobenzene, or a combination thereof, or a combination thereof with water.

In some embodiments, the solvent is a polar aprotic solvent. In some embodiments, the polar aprotic solvent is N,N-dimethylformamide, N,N-dimethylacetamide, N-methyl-2-pyrrolidone, or a combination thereof, or a combination thereof with water.

In some embodiments, the solvent is tetrahydrofuran. In some embodiments, the solvent is tetrahydrofuran and water. In some embodiments, the solvent is tetrahydrofuran and water at a temperature of about −10° C. to about 10° C. In some embodiments, the solvent is tetrahydrofuran and water at a temperature of about 0° C.

In some embodiments, the conditions comprise tetrahydrofuran at a temperature of about 0° C. and the hydrolyzing step comprises potassium carbonate, tetrahydrofuran, and water at a temperature of about 0° C.

In some embodiments, X is chloro or bromo. In some embodiments, X is chloro.

Also provided herein is a process for preparing a compound of Formula VI, or a salt thereof:

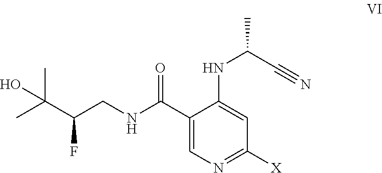

comprising contacting a compound of Formula VII, or a salt thereof;

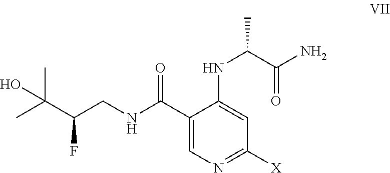

with a dehydrating agent under conditions suitable to provide a compound of Formula VI; wherein X is halo; provided that the dehydrating agent is other than trifluoroacetic anhydride.

In some embodiments, the dehydrating agent is a cyanuric chloride, a carboxylic acid anhydride, a sulfonic acid anhydride, an alkyl chloroformate, phosphorus oxychloride, phosphorus pentoxide, thionyl chloride, phosphoryl chloride, a sulfonyl chloride, oxalyl chloride, aluminum trichloride, or a dichlorophosphate. In some embodiments, the dehydrating agent is cyanuric chloride, a carboxylic acid anhydride (such as acetic anhydride), a sulfonic acid anhydride (such as trifluoromethanesulfonic anhydride), an alkyl chloroformate (such as ethyl chloroformate), phosphorus oxychloride, or phosphorus pentoxide. In some embodiments, the dehydrating agent is acetic anhydride, trifluoromethanesulfonic anhydride, ethyl chloroformate, phosphorus oxychloride, phosphorus pentoxide, thionyl chloride, phosphoryl chloride, methanesulfonyl chloride, para-toluenesulfonyl chloride, oxalyl chloride, aluminum trichloride, or ethyl dichlorophosphate.

In some embodiments, the conditions further comprise a base.

In some embodiments, the base is an amine base. In some embodiments, the amine base is N,N-diisopropylamine, triethylamine, 1,8-diazabicyclo[5.4.0]undec-7-ene, or N-methylimidazole.

In some embodiments, the amine base is triethylamine.

In some embodiments, the process further comprises a hydrolyzing step.

In some embodiments, the hydrolyzing comprises an inorganic base or an alkoxide base.

In some embodiments, the inorganic base is sodium hydroxide, lithium hydroxide, potassium hydroxide, calcium hydroxide, magnesium hydroxide, tetramethylammonium hydroxide, tetrabutylammonium hydroxide, sodium carbonate, lithium carbonate, potassium carbonate, calcium carbonate, magnesium carbonate, tetramethylammonium carbonate, tetrabutylammonium carbonate, sodium phosphate mono/di/tribasic, lithium phosphate mono/di/tribasic, potassium phosphate mono/di/tribasic, calcium phosphate mono/di/tribasic, magnesium phosphate mono/di/tribasic, tetramethylammonium phosphate mono/di/tribasic, or tetrabutylammonium phosphate mono/di/tribasic.

In some embodiments, the alkoxide base is sodium methoxide, lithium methoxide, potassium methoxide, magnesium methoxide, calcium methoxide, sodium ethoxide, lithium ethoxide, potassium ethoxide, magnesium ethoxide, calcium ethoxide, sodium tert-butoxide, lithium tert-butoxide, or potassium tert-butoxide.

In some embodiments, the conditions and hydrolyzing steps further comprise a solvent. In some embodiments, the conditions and hydrolyzing steps further comprise a solvent at a temperature of about-20° C. to about 60° C. In some embodiments, the conditions and hydrolyzing steps further comprise a solvent at a temperature of about 0° C. to about 20° C. In some embodiments, the conditions and hydrolyzing steps further comprise a solvent at a temperature of about 10° C.

In some embodiments, the solvent is an ester, an ether, a ketone, a nitrile, a hydrocarbon, a halogenated solvent, or a polar aprotic solvent. In some embodiments, the solvent is ethyl acetate, isopropyl acetate, 2-methyltetrahydrofuran, tert-butyl methyl ether, acetone, 2-butanone, 4-methyl-2-pentanone, toluene, n-heptane, dichloromethane, 1,2-dichloroethane, chlorobenzene, acetonitrile, N,N-dimethylformamide, N,N-dimethylacetamide, or N-methyl-2-pyrrolidone.

In some embodiments, the solvent is an ester. In some embodiments, the ester is ethyl acetate or isopropyl acetate.

In some embodiments, the solvent is an ether. In some embodiments, the ether is tetrahydrofuran, 2-methyltetrahydrofuran, or tert-butyl methyl ether.

In some embodiments, the solvent is a ketone. In some embodiments, the ketone is acetone, 2-butanone, or 4-methyl-2-pentanone.

In some embodiments, the solvent is a nitrile. In some embodiments, the nitrile is acetonitrile.

In some embodiments, the solvent is a hydrocarbon. In some embodiments, the hydrocarbon is toluene or n-heptane.

In some embodiments, the solvent is a halogenated solvent. In some embodiments, the halogenated solvent is dichloromethane, 1,2-dichloroethane, or chlorobenzene.

In some embodiments, the solvent is a polar aprotic solvent. In some embodiments, the polar aprotic solvent is N,N-dimethylformamide, N,N-dimethylacetamide, or N-methyl-2-pyrrolidone.

In some embodiments, the solvent is acetonitrile. In some embodiments, the solvent is acetonitrile at a temperature of about 0° C. to about 20° C. In some embodiments, the solvent is acetonitrile at a temperature of about 10° C.

In some embodiments, the conditions comprise triethylamine and acetonitrile at a temperature of about 10° C.

Also provided herein is a process for preparing a compound of Formula IX, or a salt thereof:

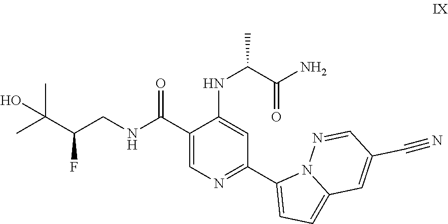 [SMILES: C[C@@H](Nc1cc(-c2ccc3cc(C#N)cnn23)ncc1C(=O)NC[C@@H](F)C(C)(C)O)C(N)=O]

comprising contacting a compound of Formula VII, or a salt thereof;

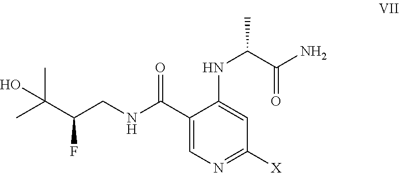

with a compound of Formula VA:

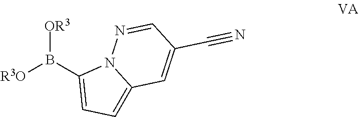

under conditions suitable to provide a compound of Formula IX; wherein X is halo; and
each R³ is independently hydrogen or C₁₋₆ alkyl, or two R³ cyclize to form a cyclic boronate ester.

In some embodiments, two R³ cyclize to form a cyclic boronate ester. In some embodiments, the moiety

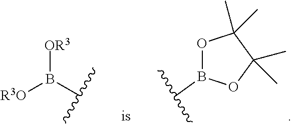

In some embodiments, X is chloro.

In some embodiments, the conditions comprise a base.

In some embodiments, the base is an inorganic base, an alkoxide base, or an organic base, such as, but not limited to, an amine base, e.g., a tertiary amine, or a nitrogen-containing heterocyclic or nitrogen-containing heteroaryl base. In some embodiments, the base is sodium hydroxide, lithium hydroxide, potassium hydroxide, calcium hydroxide, magnesium hydroxide, tetramethylammonium hydroxide, tetrabutylammonium hydroxide, sodium carbonate, lithium carbonate, potassium carbonate, calcium carbonate, magnesium carbonate, tetramethylammonium carbonate, tetrabutylammonium carbonate, sodium phosphate mono/di/tribasic, lithium phosphate mono/di/tribasic, potassium phosphate mono/di/tribasic, calcium phosphate mono/di/tribasic, magnesium phosphate mono/di/tribasic, tetramethylammonium phosphate mono/di/tribasic, tetrabutylammonium phosphate mono/di/tribasic, sodium methoxide, lithium methoxide, potassium methoxide, magnesium methoxide, calcium methoxide, sodium ethoxide, lithium ethoxide, potassium ethoxide, magnesium ethoxide, calcium ethoxide, sodium tert-butoxide, lithium tert-butoxide, potassium tert-butoxide. N-methylmorpholine, triethylamine, N-methylmorpholine, triethylamine, pyridine, or N-methylimidazole.

In some embodiments, the base is an inorganic base. In some embodiments, the inorganic base is sodium hydroxide, lithium hydroxide, potassium hydroxide, calcium hydroxide, magnesium hydroxide, tetramethylammonium hydroxide, tetrabutylammonium hydroxide, sodium carbonate, lithium carbonate, potassium carbonate, calcium carbonate, magnesium carbonate, tetramethylammonium carbonate, tetrabutylammonium carbonate, sodium phosphate mono/di/tribasic, lithium phosphate mono/di/tribasic, potassium phosphate mono/di/tribasic, calcium phosphate mono/di/tribasic, magnesium phosphate mono/di/tribasic, tetramethylammonium phosphate mono/di/tribasic, or tetrabutylammonium phosphate mono/di/tribasic.

In some embodiments, the base is an alkoxide base. In some embodiments, the alkoxide base is sodium methoxide, lithium methoxide, potassium methoxide, magnesium methoxide, calcium methoxide, sodium ethoxide, lithium ethoxide, potassium ethoxide, magnesium ethoxide, calcium ethoxide, sodium tert-butoxide, lithium tert-butoxide, or potassium tert-butoxide.

In some embodiments, the base is an amine base. In some embodiments, the amine base is triethylamine, diisopropylethyl amine, tributyl amine, N-methylmorpholine, pyridine, 4-dimethylaminopyridine, or N-methylimidazole. In some embodiments, the base is a tertiary amine. In some embodiments, the tertiary amine is N-methylmorpholine or triethylamine.

In some embodiments, the base is a nitrogen-containing heterocyclic or nitrogen-containing heteroaryl base. In some embodiments, the nitrogen-containing heterocyclic or nitrogen-containing heteroaryl base is N-methylmorpholine, pyridine, or N-methylimidazole.

In some embodiments, the base is potassium phosphate tribasic.

In some embodiments, the conditions comprise a catalyst. In some embodiments, the conditions comprise a base and a catalyst.

In some embodiments, the catalyst is a palladium(II) salt, a palladium(0) salt, copper(II) trifluoromethanesulfonate, or a combination thereof. In some embodiments, the catalyst is a palladium(II) or palladium(0) salt. In some embodiments, the catalyst is palladium(II) 2,4-pentanedionate (Pd(acac)₂), allylpalladium(II) chloride dimer ([Pd(allyl)Cl₂]), bis(acetonitrile)dichloropalladium(II) (Pd(MeCN)₂Cl₂), tris(dibenzylideneacetone)dipalladium(0) (Pd(dba)₃), palladium(II) trifluoroacetate (Pd(TFA)₂), tetrakis(triphenylphosphine)palladium(0) (Pd(PPh₃)₄), palladium(II) chloride (PdCl₂), or a combination thereof.

In some embodiments, the catalyst is a palladium(II) salt. In some embodiments, the palladium(II) salt is palladium(II) 2,4-pentanedionate, allylpalladium(II) chloride dimer, bis(acetonitrile)dichloropalladium(II), palladium(II) trifluoroacetate, or palladium(II) chloride.

In some embodiments, the catalyst is a palladium(0) salt. In some embodiments, the palladium(0) salt is tetrakis (triphenylphosphine)palladium(0) or tris(dibenzylideneacetone)dipalladium(0).

In some embodiments, the conditions comprise a catalyst, a ligand, and a base.

In some embodiments, the catalyst is palladium(II) acetate.

In some embodiments, the ligand is a dialkylbiaryl phosphine ligand, a monodentate phosphine ligand, or a bidentate phosphine ligand. In some embodiments, the ligand is 2-di-tert-butylphosphino-2′,4′,6′-triisopropylbiphenyl (t-BuXPhos), 2-dicyclohexylphosphino-2′,6′-dimethoxybiphenyl (SPhos), 2-(dicyclohexylphosphino) 3,6-dimethoxy-2′,4′,6′-triisopropyl-1,1′-biphenyl (BrettPhos), 2-dicyclohexylphosphino-2′,6′-diisopropoxy biphenyl (RuPhos), triphenylphosphine, tri-tert-butyl phosphine, tricyclohexyl phosphine, 4,5-bis(diphenylphosphino)-9,9-dimethylxanthene (XantPhos), 1,1′-bis(di-tert-butylphosphino)ferrocene (dtbpf), or 1,1′-ferrocenediyl-bis(diphenylphosphine) (dppf).

In some embodiments, the ligand is a dialkylbiaryl phosphine ligand. In some embodiments, the dialkylbiaryl phosphine ligand is 2-di-tert-butylphosphino-2′,4′,6′-triisopropylbiphenyl (t-BuXPhos), 2-dicyclohexylphosphino-2′,6′-dimethoxybiphenyl (Sphos), 2-(dicyclohexylphosphino) 3,6-dimethoxy-2′,4′,6′-triisopropyl-1,1′-biphenyl (BrettPhos), or 2-dicyclohexylphosphino-2′,6′-diisopropoxy biphenyl (RuPhos).

In some embodiments, the ligand is a monodentate phosphine ligand. In some embodiments, the monodentate phosphine ligand is triphenylphosphine, tri-tert-butyl phosphine, or tricyclohexyl phosphine.

In some embodiments, the ligand is a bidentate phosphine ligand. In some embodiments, the bidentate phosphine ligand is 4,5-bis(diphenylphosphino)-9,9-dimethylxanthene (XantPhos), 1,1′-bis(di-tert-butylphosphino)ferrocene (dtbpf), or 1,1′-ferrocenediyl-bis (diphenylphosphine) (dppf).

In some embodiments, the ligand is 2-dicyclohexylphosphino-2′,4′,6′-triisopropylbiphenyl (Xphos).

In some embodiments, the catalyst is a precatalyst, such as a precatalyst comprising a preformed mixture of a palladium catalyst and ligand, such as, but not limited to, those listed hereinabove, as well as other Pd precatalysts (such as, e.g., 2-dicyclohexylphosphino-2′,4′,6′-triisopropyl-1,1′-biphenyl)[2-(2′-amino-1,1′-biphenyl)]palladium(II) methanesulfonate).

In some embodiments, the conditions further comprise a solvent. In some embodiments, the conditions further comprise a solvent at a temperature of about 45° C. to about 110° C. In some embodiments, the conditions further comprise a solvent at a temperature of about 70° C. to about 80° C. In some embodiments, the conditions further comprise a solvent at a temperature of about 75° C.

In some embodiments, the solvent is an ether, a ketone, a polar aprotic solvent, a hydrocarbon, a nitrile, or a combination thereof, or a combination thereof with water. In some embodiments, the solvent is tetrahydrofuran, tert-butyl methyl ether, cyclopentyl methyl ether (CPME), acetone, 2-butanone, 4-methyl-2-pentanone, N,N-dimethylformamide, N,N-dimethylacetamide, N-methyl-2-pyrrolidone, toluene, n-heptane, acetonitrile, or a combination thereof, or a combination thereof with water.

In some embodiments, the solvent is an ether. In some embodiments, the solvent is 2-methyltetrahydrofuran and water, and the temperature is from about 70° C. to about 80° C., or about 75° C.

Also provided herein is a process for preparing Compound I, or a salt thereof:

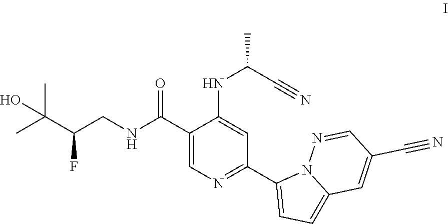 [SMILES: C[C@H](C#N)Nc1cc(-c2ccc3cc(C#N)cnn23)ncc1C(=O)NC[C@@H](F)C(C)(C)O]

comprising contacting a compound of Formula IX, or a salt thereof:

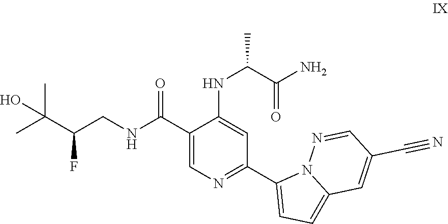 [SMILES: C[C@@H](Nc1cc(-c2ccc3cc(C#N)cnn23)ncc1C(=O)NC[C@@H](F)C(C)(C)O)C(N)=O]

under conditions suitable to provide Compound I.

In some embodiments, the conditions comprise a dehydrating agent as described herein.

In some embodiments, the dehydrating agent is a cyanuric chloride, a carboxylic acid anhydride, a sulfonic acid anhydride, an alkyl chloroformate, phosphorus oxychloride, or phosphorus pentoxide.

In some embodiments, the dehydrating agent is acetic anhydride, trifluoroacetic anhydride, trifluoromethanesulfonic anhydride, ethyl chloroformate, phosphorus oxychloride, or phosphorus pentoxide.

In some embodiments, the dehydrating agent is trifluoroacetic anhydride.

In some embodiments, the conditions comprise a dehydrating agent and a base, such as an amine base. In some embodiments, the amine base is N-methylimidazole, N,N-diisopropylamine, triethylamine, N,N-diisopropylethylamine, 1,8-diazabicyclo[5.4.0]undec-7-ene, or N-methylmorpholine.

In some embodiments, the conditions comprise trifluoroacetic anhydride and pyridine.

In some embodiments, the conditions comprise a dehydrating agent, a base, and a solvent.

In some embodiments, the solvent is an ester, ether, ketone, nitrile, hydrocarbon, a halogenated solvent, or polar aprotic solvent, or a mixture thereof.

In some embodiments, the solvent is ethyl acetate, isopropyl acetate, tetrahydrofuran, 2-methyltetrahydrofuran, tert-butyl methyl ether, acetone, 2-butanone, 4-methyl-2-pentanone, acetonitrile, toluene, n-heptane, dichloromethane, 1,2-dichloroethane, chlorobenzene, N,N-dimethylformamide, N,N-dimethylacetamide, N-methyl-2-pyrrolidone, or a mixture thereof.

In some embodiments, the conditions comprise trifluoroacetic anhydride and pyridine in tetrahydrofuran.

In some embodiments, the conditions comprise a temperature of about −20° C. to about 20° C. In some embodiments, the conditions comprise a temperature of about-10° C. to about 10° C. In some embodiments, the conditions comprise a temperature of about 0° C.

In some embodiments, the conditions comprise a hydrolysis step.

In some embodiments, the conditions comprise a dehydrating agent, and optional hydrolysis step. In some embodiments, the conditions comprise a dehydrating agent followed by a hydrolysis step.

In some embodiments, the hydrolysis step comprises a base.

In some embodiments, the base is an inorganic base or an alkoxide base. In some embodiments, the base is sodium hydroxide, lithium hydroxide, potassium hydroxide, calcium hydroxide, magnesium hydroxide, tetramethylammonium hydroxide, tetrabutylammonium hydroxide, sodium carbonate, lithium carbonate, potassium carbonate, calcium carbonate, magnesium carbonate, tetramethylammonium carbonate, tetrabutylammonium carbonate, sodium phosphate tribasic, sodium phosphate dibasic, sodium phosphate monobasic, lithium phosphate tribasic, lithium phosphate dibasic, lithium phosphate monobasic, potassium phosphate tribasic, potassium phosphate dibasic, potassium phosphate monobasic, calcium phosphate tribasic, calcium phosphate dibasic, calcium phosphate monobasic, magnesium phosphate tribasic, magnesium phosphate dibasic, magnesium phosphate monobasic, tetraalkylammonium phosphate tribasic, tetraalkylammonium phosphate dibasic, tetraalkylammonium phosphate monobasic, sodium methoxide, lithium methoxide, potassium methoxide, magnesium methoxide, calcium methoxide, sodium ethoxide, lithium ethoxide, potassium ethoxide, magnesium ethoxide, calcium ethoxide, sodium tert-butoxide, lithium tert-butoxide, potassium tert-butoxide, magnesium tert-butoxide, calcium tert-butoxide, or a mixture thereof.

In some embodiments, the base is sodium carbonate.

In some embodiments, the hydrolysis step comprises a solvent, such as, but not limited to, esters (such as, but not limited to, ethyl acetate, isopropyl acetate, and the like), ethers (such as, but not limited to, 2-methyltetrahydrofuran, tert-butyl methyl ether, and the like), or ketones (such as, but not limited to, acetone, 2-butanone, and the like).

In some embodiments, the conditions comprise a temperature of about −20° C. to about 60° C. In some embodiments, the conditions comprise a temperature of about −10° C. to about 10° C. In some embodiments, the conditions comprise a temperature of about 0° C.

### Compounds

In certain embodiments, the disclosure provides for intermediate compounds that are useful in the processes described herein.

It can be appreciated that the straight bolded or dashed bond is used to indicate relative stereochemistry, and the wedged bolded or dashed bond is used to indicate absolute stereochemistry. Where the composition is identified as enantiomerically enriched, it is intended that the composition comprises more than 50% of a single enantiomer, or at least about 55%, or at least about 60%, or at least about 65%, or at least about 70%, or at least about 75%, or at least about 80%, or at least about 85%, or at least about 90%, or at least about 95%, or at least about 97%, or about 99% ee.

Provided herein is a citric acid salt of Compound I:

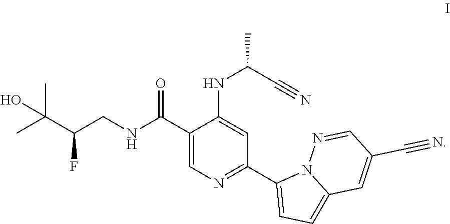 [SMILES: C[C@H](C#N)Nc1cc(-c2ccc3cc(C#N)cnn23)ncc1C(=O)NC[C@@H](F)C(C)(C)O]

Provided herein is a citric acid salt of Compound I:

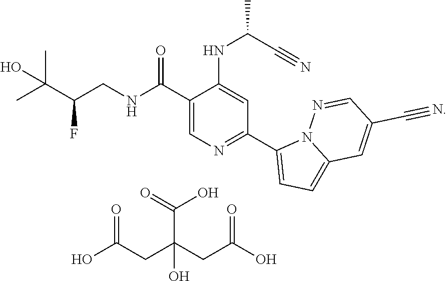

In certain embodiments, the citric acid salt of Compound I is the monocitrate salt of Compound I.

Also provided is a methanesulfonic acid salt of Compound II:

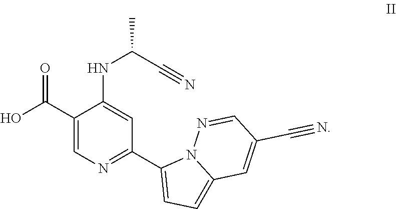 [SMILES: C[C@H](C#N)Nc1cc(-c2ccc3cc(C#N)cnn23)ncc1C(=O)O]

Also provided is a methanesulfonic acid salt of Compound II:

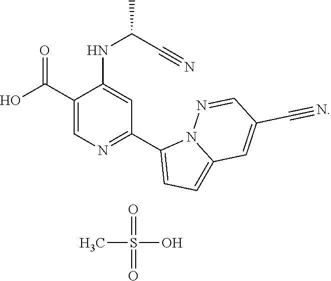

Also provided is a compound of Formula IIA:

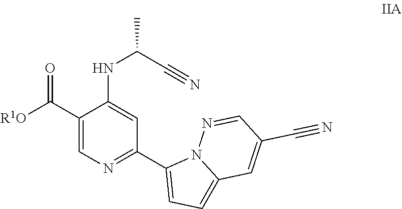

wherein R′ is a protecting group.

In some embodiments, R′ is a carboxy protecting group. In certain embodiments, the carboxy protecting group is C₁₋₆ alkyl optionally substituted with C₁₋₆ alkoxy, cyano, —Si(C₁₋₆ alkyl)₃, or aryl, wherein the aryl is optionally substituted with one or more substituents independently selected from halo, nitro, C₁₋₆ alkyl, and C₁₋₆ alkoxy. In certain embodiments, R¹ is methyl, ethyl, n-propyl, isopropyl, n-butyl, isobutyl, tert-butyl, methoxymethyl, t-butoxymethyl, benzyl, p-methoxybenzyl, or p-nitrobenzyl.

In some embodiments, R¹ is methyl, ethyl, n-propyl, isopropyl, n-butyl, isobutyl, sec-butyl, tert-butyl, or benzyl. In some embodiments, R¹ is methyl, ethyl, tert-butyl, or benzyl.

In some embodiments, R¹ is C₁₋₆ alkyl. In some embodiments, R¹ is methyl, ethyl, or tert-butyl. In some embodiments, R¹ is tert-butyl.

Also provided is a compound of Formula IV:

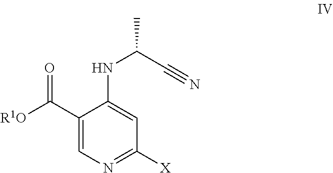

wherein R¹ and X are each independently as defined herein.

In some embodiments, R¹ is C₁₋₆ alkyl. In some embodiments, X is halo. In some embodiments, X is chloro or bromo. In some embodiments, X is chloro.

Also provided is a compound of Formula V:

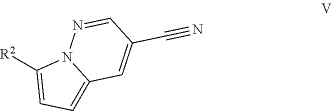

wherein R² is hydrogen or —B(OR³)₂; and
and each R³ is independently hydrogen or C₁₋₆ alkyl, or two R³ cyclize to form a cyclic boronate ester.

In some embodiments, R² is hydrogen.

In some embodiments, each R³ is independently hydrogen or C₁₋₆ alkyl. In some embodiments, each R³ is hydrogen. In some embodiments, each R³ is C₁-6 alkyl. In some embodiments, two R³ cyclize to form a cyclic boronate ester. In some embodiments, two R³ cyclize to form a 5-10 membered cyclic boronate ester. In some embodiments, R² is

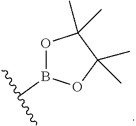 [SMILES: *B1OC(C)(C)C(C)(C)O1]

Also provided is a compound, or a salt thereof, selected from:

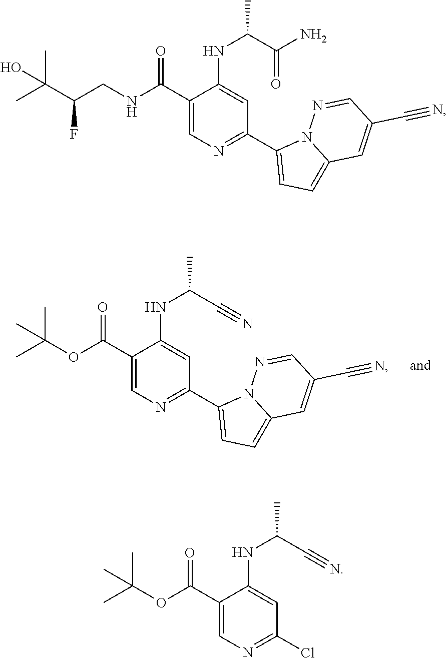 [SMILES: C[C@@H](Nc1cc(-c2ccc3cc(C#N)cnn23)ncc1C(=O)NC[C@@H](F)C(C)(C)O)C(N)=O ; C[C@H](C#N)Nc1cc(-c2ccc3cc(C#N)cnn23)ncc1C(=O)OC(C)(C)C ; C[C@H](C#N)Nc1cc(Cl)ncc1C(=O)OC(C)(C)C]

Also provided is a compound having the structure:

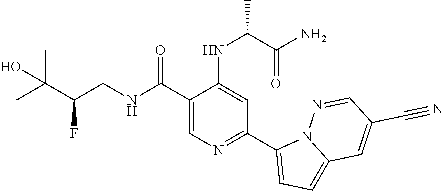 [SMILES: C[C@@H](Nc1cc(-c2ccc3cc(C#N)cnn23)ncc1C(=O)NC[C@@H](F)C(C)(C)O)C(N)=O]

or a salt thereof.

Also provided is a compound having the structure:

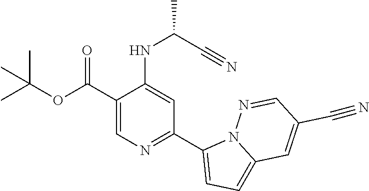 [SMILES: C[C@H](C#N)Nc1cc(-c2ccc3cc(C#N)cnn23)ncc1C(=O)OC(C)(C)C]

or a salt thereof.

Also provided is a compound having the structure:

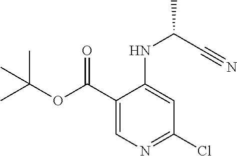 [SMILES: C[C@H](C#N)Nc1cc(Cl)ncc1C(=O)OC(C)(C)C]

or a salt thereof.

It is appreciated that certain features described herein, which are, for clarity, described in the context of separate embodiments, may also be provided in combination in a single embodiment.

Conversely, various features described herein, which are, for brevity, described in the context of a single embodiment, may also be provided separately or in any suitable subcombination.

### EXAMPLES

The compounds of the disclosure may be prepared using methods disclosed herein and routine modifications thereof which will be apparent given the disclosure herein and methods well known in the art. Conventional and well-known synthetic methods may be used in addition to the teachings herein. The synthesis of compounds described herein may be accomplished as described in the following examples. If available, reagents may be purchased commercially, e.g., from Sigma Aldrich or other chemical suppliers. Unless otherwise noted, the starting materials for the following reactions may be obtained from commercial sources.

### LIST OF ABBREVIATIONS AND ACRONYMS

Abbreviation Meaning® C. degrees Celsius
DMSO dimethylsulfoxide
EtOH ethanol
HCl hydrochloric acid
KHCO₃ potassium bicarbonate
K₂CO₃ potassium carbonate
K₃PO₄ tripotassium phosphate
MeCN acetonitrile
MeOH methanol
MTBE methyl tert-butyl ether
MIBK methyl isobutyl ketone
NaOH sodium hydroxide
Nf perfluorobutanesulfonyl
Pd/C palladium on carbon
RuPhos 2-dicyclohexylphosphino-2′,6′-diisopropoxybiphenyl
TCFH N,N,N′,N′-tetramethylchloroformamidinium hexafluorophosphate
TEA triethylamine
TFAA trifluoroacetic acid
THF tetrahydrofuran
XPhos dicyclohexyl[2′,4′,6′-tris(propan-2-yl)[1,1′-biphenyl]-2-yl]phosphane

### Procedure A: Synthesis of (R)-4-amino-3-fluoro-2-methylbutan-2-ol hydrochloride

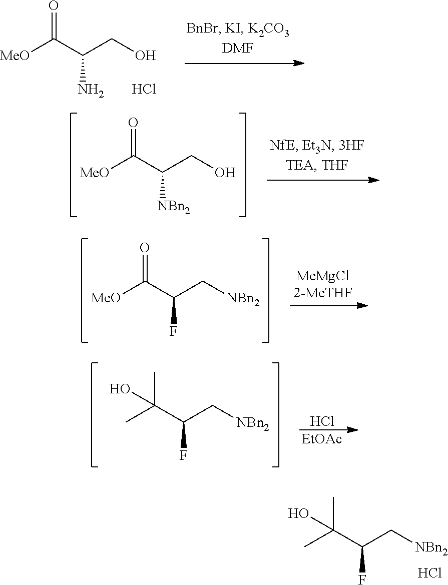

A reactor was charged with methyl L-serinate hydrochloride (1.0 equiv, scaling factor), N,N-dimethylformamide (8.0 volumes), potassium carbonate (2.0 equiv), and potassium iodide (0.5 equiv). The temperature was adjusted to about 5° C. and benzyl bromide (1.95 equiv) was added over about 4 hours maintaining the temperature at about 5° C. The mixture was warmed to about 25° C. and agitated for about 16 h. The mixture was cooled to about 5° C. and tert-butylmethyl ether (6.0 volumes) and water (10.0 volumes) were charged. The mixture was warmed to about 25° C. and agitated for about 2 hours. The aqueous layer was removed and the organic layer was washed with water (5.0 volumes) and twice with 10% w/w sodium sulfate in water (4.0 volumes each). The mixture was concentrated at about 50° C. to about 3 volumes and solvent-swapped to tetrahydrofuran (8.0 volumes) to give a solution of methyl dibenzyl-L-serinate.

A reactor was charged with the methyl dibenzyl-L-serinate solution in tetrahydrofuran (1.0 equiv. scaling factor) and the temperature was adjusted to about 25° C. The reactor was then charged with triethylamine trihydrofluoride (1.3 equiv) over about 2 hours, followed by diisopropylethyl amine (2.8 equiv) over about 2 hours maintaining the temperature at about 25° C. The reactor was charged with perfluorobutanesulfonyl fluoride (1.3 equiv) over about 3 hours maintaining the temperature at about 25° C. The reaction mixture was adjusted to about 40° C. and the reaction was agitated for about 15 hours. The reaction mixture was adjusted to about 20° C. over about 2 hours. To the mixture was added water (5.0 volumes) maintaining the temperature at about 20° C. The mixture was charged with methylcyclohexane (4.0 volumes) and agitated at about 25° C. for about 1 hour. Agitation was stopped to give three layers. The upper layer was removed and the lower two layers returned to the reactor and charged with methylcyclohexane (4.0 volumes) and agitated at about 25° C. for about 1 hour. Agitation was stopped to give three layers. The upper and lower layers were removed and middle layer was returned to the reactor and washed with 7% w/w potassium bicarbonate in water (4.5 volumes) and water (4.0 volumes). The mixture was concentrated to about 3 volumes and solvent-swapped to 2-methyltetrahydrofuran (7.0 volumes) to provide a solution of methyl (R)-3-(dibenzylamino)-2-fluoropropanoate.

A reactor was charged with the methyl (R)-3-(dibenzylamino)-2-fluoropropanoate solution in 2-methyltetrahydrofuran (1.0 equiv. scaling factor) and cooled to about −30° C. A 3M methylmagnesium chloride solution in tetrahydrofuran (2.4 equiv) was added over about 5 hours maintaining the temperature at about −30° C. The mixture was agitated for about 6 hours. In a separate reactor was charged 1N aqueous HCl (7.0 volumes) and cooled to about 5° C. The mixture from the first reactor was added to the HCl solution over about 2 hours maintaining the temperature at about 5° C. The mixture was warmed to about 25° C. and agitated for about 1 hour. The layers were separated and the organic layer extracted twice with 1N aqueous HCl (6.0 volumes and then 3.0 volumes). The two acidic extractions were combined and potassium carbonate was added until about pH 9. This basic aqueous layer was extracted with ethyl acetate (5.0 volumes), the aqueous layer removed and the organic mixture concentrated to about 3 volumes at about 45° C. The mixture was azcotropically dried with successive ethyl acetate (6.0 volumes) additions and concentrated to about 3 volumes at about 45° C. Ethyl acetate (6.0) volumes was added to provide a solution of (R)-4-(dibenzylamino)-3-fluoro-2-methylbutan-2-ol.

A reactor was charged with ethyl acetate (2.0 volumes) and adjusted to about −10° C. Hydrogen chloride gas was passed into the ethyl acetate for about 2 hours. In a separate reactor, the (R)-4-(dibenzylamino)-3-fluoro-2-methylbutan-2-ol solution in ethyl acetate (1.0 equiv. scaling factor) was charged and adjusted to about 25° C. The hydrogen chloride solution in ethyl acetate was added over about 30 minutes. The mixture was agitated at about 25° C. for about 12 hours. The mixture was then degassed by passing nitrogen into the mixture for about 1 hour. The slurry was filtered and washed twice with ethyl acetate (1.0 volume) and dried at about 45° C. for about 20 hours to give (R)-4-(dibenzylamino)-3-fluoro-2-methylbutan-2-ol hydrochloride.

¹H NMR (400 MHZ, MeOD-d₄) δ 7.51-7.56 (m, 10H), 4.65 (ddd, J=2.6, 7.4, 47.9 Hz, 1H), 4.37-4.56 (bm, 4H), 3.47-3.66 (m, 2H), 1.17 (s, 3H), 1.03 (s, 3H) ppm.

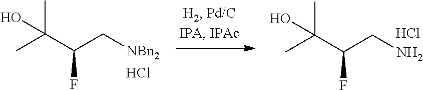

A reactor was charged with (R)-4-(dibenzylamino)-3-fluoro-2-methybutan-2-ol hydrochloride (1.0 equiv), isopropyl alcohol (8.0 volumes), and 10% wet Pd/C (about 7% w/w). The reactor was flushed three times with hydrogen and the mixture was heated to about 45° C. The reactor was pressurized with hydrogen (about 45 psi) and agitated at about 45° C. for about 20 hours. The mixture was adjusted to about 25° C. and filtered over diatomaceous earth, washing the cake twice with isopropyl alcohol (2.0 volumes each). The mixture was concentrated under vacuum below about 50° C. to about 2 volumes. The mixture was azeotropically dried with successive charges of isopropyl alcohol (4.0 volumes) and concentrating at about 50° C. to about 2 volumes. The reactor was charged with isopropyl alcohol (2.0 volumes). The mixture was adjusted to about 45° C. over about 1 hour and agitated for about 8 hours. The mixture was adjusted to about 25° C. over about 4 hours and isopropyl acetate (5.0 volumes) was charged over about 2 hours. The mixture was agitated at about 25° C. for about 20 hours. The mixture was filtered and the filtrate was agitated at about 25° C. for about 4 hours forming a second crop that was filtered with the first crop. The cake was washed with a 5:1 mixture of isopropyl acetate:isopropyl alcohol (1.0 volume) and dried at about 45° C. for about 20 h to give (R)-4-amino-3-fluoro-2-methylbutan-2-ol hydrochloride.

¹H NMR (400 MHZ, DMSO-d₆) § 8.41 (s, 3H), 5.03 (s, 1H), 4.42-4.57 (m, 1H), 2.97-3.20 (m, 2H), 1.12-1.13 (d, J=4.0 Hz, 6H) ppm.

### Procedure B: Synthesis of pyrrolo[1,2-b]pyridazine-3-carbonitrile

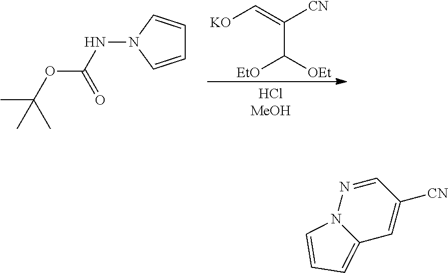

A reactor was charged with potassium (Z)-2-cyano-3,3-diethoxyprop-1-en-1-olate solution in methanol (1.1 equiv) and concentrated aqueous HCl (1.7 equiv). tert-Butyl (1H-pyrrol-1-yl) carbamate (1.0 equiv, scaling factor) was added to the reaction mixture at about 25° C. The mixture was agitated for about 2 hours at about 25° C. Water (4.5 volumes) was added to the reactor and the pH of the mixture was adjusted to about pH 8-9 with 2N NaOH solution to form a slurry. The mixture was agitated for about 30 minutes at about 25° C. The slurry was filtered and the filter cake washed with water (1.3 volumes). The wet cake was transferred to a reactor and slurried in water (3.0 volumes) for about 2 hours at about 25° C. The slurry was filtered, the cake washed was with water (1.0 volume), and dried at about 40° C. to give pyrrolo[1,2-b]pyridazine-3-carbonitrile.

¹H NMR (400 MHZ, Chloroform-d) δ 8.03-8.16 (m, 2H), 7.93 (ddd, J=2.6, 1.4, 0.6 Hz, 1H), 7.04 (dd, J=4.5, 2.7 Hz, 1H), 6.84 (dd, J=4.6, 1.4 Hz, 1H) ppm.

### Procedure C: Synthesis of 7-bromopyrrolo[1,2-b]pyridazine-3-carbonitrile

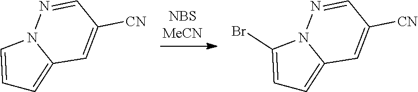 [SMILES: N#Cc1cnn2cccc2c1 ; N#Cc1cnn2c(Br)ccc2c1]

A reactor was charged with pyrrolo[1,2-b]pyridazine-3-carbonitrile (1.0 equiv, scaling factor) and acetonitrile (20 volumes). A separate reactor was charged with N-bromosuccinimide (1.1 equiv) and acetonitrile (12 volumes). The N-bromosuccinimide solution was added to the first reactor over about 9 hours at about 15° C., forming a slurry. The mixture was aged about 30 minutes at about 15° C. and was then cooled to about 5° C. Water (33 volumes) was charged over about 6 hours at about 5° C. and aged about 1 hour at about 5° C. The slurry was filtered and the cake was washed with water (2.0 volumes) and dried at about 50° C. to give 7-bromopyrrolo[1,2-b]pyridazine-3-carbonitrile.

¹H NMR (400 MHZ, Chloroform-d) § 8.28 (d, J=2.1 Hz, 1H), 8.10 (d, J=2.1 Hz, 1H), 7.12 (d, J=4.8 Hz, 1H), 6.93 (d, J=4.8 Hz, 1H) ppm.

### Procedure D: Synthesis of 7-(4,4,5,5-tetramethyl-1,3,2-dioxaborolan-2-yl)pyrrolo[1,2-b]pyridazine-3-carbonitrile

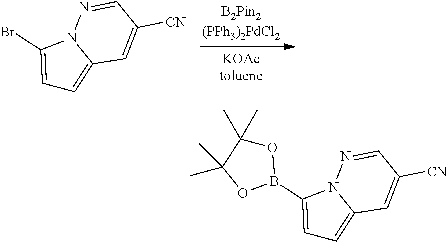 [SMILES: N#Cc1cnn2c(Br)ccc2c1 ; CC1(C)OB(c2ccc3cc(C#N)cnn23)OC1(C)C]

A reactor was charged with toluene (10.0 volumes) and heated to reflux for about 11 hours to with a Dean-Stark trap to azeotropically dry. The contents were cooled to about 20° C. and degassed with nitrogen for about 2 h. 7-Bromopyrrolo[1,2-b]pyridazine-3-carbonitrile (1.0 equiv, scaling factor) was charged to the reactor, followed by pinacol diboron (1.2 equiv) and potassium acetate (3.0 equiv). The mixture was degassed with nitrogen for about 1 hour. The mixture was heated to reflux with a Dean-Stark trap for about 2 hours to azeotropically dry, and then cooled to about 20° C. To the mixture was charged bis (triphenylphosphine)palladium(II) chloride (0.02 equiv.) and the mixture was heated to about 100° C. for about 16 h. The mixture was cooled to about 25° C. and n-heptane (6.0 volumes) was charged and the mixture was agitated for about 2 h, forming a slurry. The slurry was filtered over celite and activated charcoal and washed with a 2:1 mixture of toluene:n-heptane (6.0 volumes). The mixture was solvent swapped to n-heptane (5.0 volumes). The slurry was adjusted to about 30° C. and filtered, washing the cake with n-heptane (1.0 volume). A reactor was charged with the wet cake, MTBE (1.0 volume) and n-heptane (5.0 volumes). The slurry was agitated for about 1 hour at about 70° C., and about 1 hour at about 30° C. The slurry was filtered and washed with n-heptane (1.0 volume). A reactor was charged with the wet cake, MTBE (1.0 volume) and n-heptane (5.0 volumes). The slurry was agitated for about 1 hour at about 70° C., and about 1 hour at about 30° C. The slurry was filtered and washed with n-heptane (1.0 volume). The cake was dried at about 50° C. to give 7-(4,4,5,5-tetramethyl-1,3,2-dioxaborolan-2-yl)pyrrolo[1,2-b]pyridazine-3-carbonitrile.

¹H NMR (400 MHZ, Chloroform-d) δ 8.31 (d, J=2.3 Hz, 1H), 8.14 (d, J=2.2 Hz, 1H), 7.52 (d, J=4.6 Hz, 1H), 6.84 (d, J=4.6 Hz, 1H), 1.41 (s, 12H) ppm.

### Procedure E: Synthesis of tert-butyl (R)-6-chloro-4-((1-cyanoethyl)amino)nicotinate

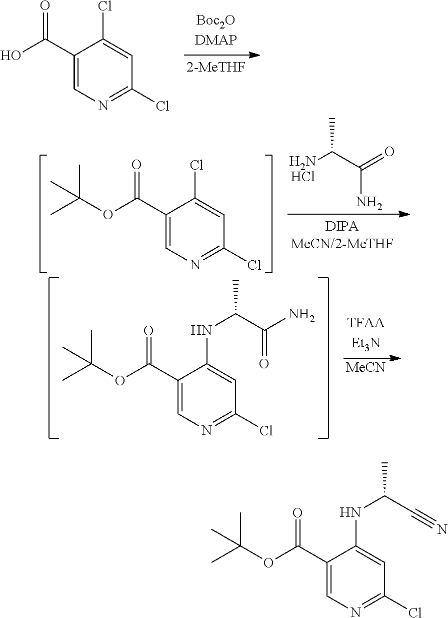

A reactor was charged with 4,6-dichloronicotinic acid (1.0 equiv, scaling factor), di-tert-butyl dicarbonate (1.2 equiv), and 2-methyltetrahydrofuran (6.0 volumes). The mixture was warmed to about 60° C. and 4-dimethylaminopyridine (0.05 equiv) in 2-methyltetrahydrofuran (1.0 volumes) was charged. After aging the reaction mixture for about 2 hours, water (5.0 volumes) was charged and the mixture was agitated about 15 minutes. The lower aqueous layer was removed and the was washed with water (6.0 volumes). The contents of the reactor were concentrated to about 3 volumes and then charged with 2-methyltetrahydrofuran (10.0 volumes). The mixture was concentrated to about 3 volumes at about 50° C. and was charged with acetonitrile (2.0 volumes) to give tert-butyl 4,6-dichloronicotinate.

A reactor was charged with the tert-butyl 4,6-dichloronicotinate solution in 2-methyltetrahydrofuran and acetonitrile (1.0 equiv, scaling factor), (R)-2-aminopropanamide hydrochloride (2.5 equiv) and N,N-diisopropylamine (4.5 equiv). The reaction mixture was warmed to about 80° C. for about 18 hours. The resulting slurry was cooled to about 60° C. and washed twice with water (2.5 volumes and then 2.0 volumes). Acetonitrile (10.0 volumes) was charged to the mixture and concentrated at about 50° C. to about 2 volumes to give tert-butyl (R)-4-((1-amino-1-oxopropan-2-yl)amino)-6-chloronicotinate.

¹H NMR (400 MHZ, DMSO-d₆) δ 8.50 (s, 1H), 8.44 (d, J=7.0 Hz, 1H), 7.62 (s, 1H), 7.31 (s, 1H), 6.57 (s, 1H), 4.16 (p, J=6.8 Hz, 1H), 1.55 (s, 9H), 1.36 (d, J=6.7 Hz, 3H) ppm.

¹³C NMR (101 MHZ, DMSO-d₆) δ 173.15, 165.78, 154.30, 154.12, 152.66, 108.12, 104.86, 82.04, 50.41, 27.81, 18.49 ppm.

A reactor was charged with the tert-butyl (R)-4-((1-amino-1-oxopropan-2-yl)amino)-6-chloronicotinate solution in acetonitrile (1.0 equiv, scaling factor) and triethylamine (1.0 equiv) and the mixture was cooled to about 10° C. Trifluoroacetic anhydride (2.1 equiv) was charged over about 30 minutes and the reaction was allowed to stir for about 2 hours. A 10% w/w solution of KHCO₃ in water (15.0 volumes) was charged over about 1 hour. The slurry was filtered and the cake was washed with water (4.0 volumes) and dried under vacuum at about 50° C. to provide tert-butyl (R)-6-chloro-4-((1-cyanoethyl)amino) nicotinate.

¹H NMR (400 MHZ, DMSO-d₆) δ 8.60 (s, 1H), 8.20 (d, J=7.7 Hz, 1H), 7.07 (s, 1H), 5.01 (p, J=7.1 Hz, 1H), 1.67 (d, J=7.0 Hz, 3H), 1.56 (s, 9H) ppm.

¹³C NMR (101 MHz, DMSO-d₆) δ 166.1, 155.2, 154.3, 153.2, 119.9, 109.3, 106.4, 83.1, 37.6, 28.2, 18.6 ppm.

### Procedure F: Synthesis of tert-butyl (R)-4-((1-cyanoethyl)amino)-6-(3-cyanopyrrolo[1,2-b]pyridazin-7-yl)nicotinate

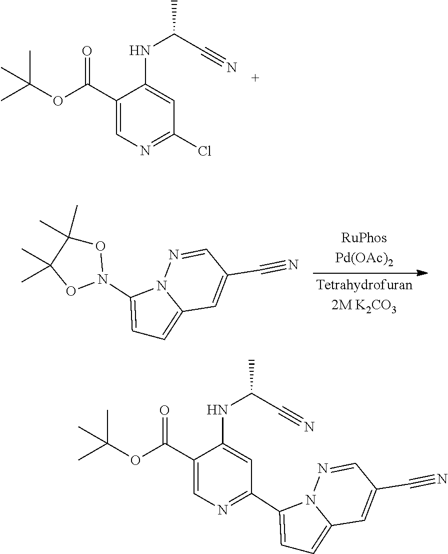 [SMILES: C[C@H](C#N)Nc1cc(Cl)ncc1C(=O)OC(C)(C)C ; CC1(C)ON(c2ccc3cc(C#N)cnn23)OC1(C)C ; C[C@H](C#N)Nc1cc(-c2ccc3cc(C#N)cnn23)ncc1C(=O)OC(C)(C)C]

A reactor was charged with tert-butyl (R)-6-chloro-4-((1-cyanoethyl)amino) nicotinate (1.0 equiv, scaling factor), 7-(4,4,5,5-tetramethyl-1,3,2-dioxaborolan-2-yl)pyrrolo[1,2-b]pyridazine-3-carbonitrile (1.3 equiv), RuPhos (0.05 equiv) and palladium(II) acetate (0.025 equiv). The reactor was inerted with nitrogen and charged with tetrahydrofuran (20.0 volumes) and 2M aqueous potassium carbonate solution (2.0 equiv). The resulting mixture was agitated under reflux for about 3 hours, and then cooled to about 40° C. To the reaction mixture was charged diatomaceous earth (150% w/w), trithiocyanuric acid trisodium hydrate (0.3 equiv), Darco G-60 (50% w/w), and 2-methyltetrahydrofuran (5.0 volumes). The resulting mixture was agitated for approximately 17 hours at approximately 40° C., then filtered. The reactor and filter cake were rinsed with water (10.0 volumes) and three times with 2-methyltetrahydrofuran (10.0 volumes). The combined filtrates were washed two times with water (10.0 volumes each) at about 40° C. The layers were separated and the organic layer was then concentrated to about 10 volumes at about 70° C. and solvent-swapped to toluene (10.0 volumes). The mixture was cooled to about 55° C. and n-heptane (12.0 volumes) was charged over about 2 hours and the resulting slurry was aged for about 1 hour at about 55° C. before cooling to about 20° C. over about 4 hours. The reaction mixture was stirred for about 16 hours at 20° C. and then filtered. The filter cake was washed twice with a mixture of 5:6 toluene:n-heptane (3.0 volumes each). The cake was dried for about 24 hours at about 50° C. to provide tert-butyl (R)-4-((1-cyanoethyl)amino)-6-(3-cyanopyrrolo[1,2-b]pyridazin-7-yl)nicotinate.

¹H NMR (400 MHZ, DMSO-d₆) δ 8.87 (s, 1H), 8.85 (d, J=2.2 Hz, 1H), 8.65 (d, J=2.2 Hz, 1H), 8.29 (s, 1H), 8.19 (d, J=7.1 Hz, 1H), 7.86 (d, J=4.8 Hz, 1H), 7.11 (d, J=4.8 Hz, 1H), 4.94 (p, J=7.0 Hz, 1H), 1.73 (d, J=6.9 Hz, 3H), 1.57 (s, 9H) ppm.

### Procedure G: Alternate Synthesis of tert-butyl (R)-4-((1-cyanoethyl)amino)-6-(3-cyanopyrrolo[1,2-b]pyridazin-7-yl)nicotinate

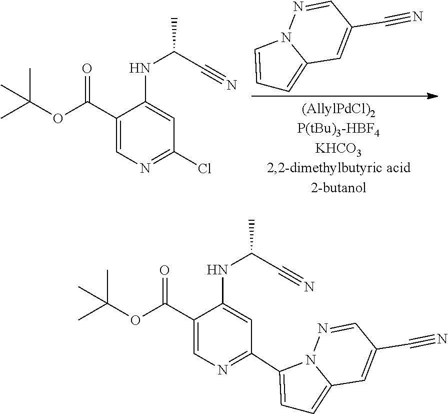

A reactor was charged with tert-butyl (R)-6-chloro-4-((1-cyanoethyl)amino) nicotinate (1.0 equiv, scaling factor), pyrrolo[1,2-b]pyridazine-3-carbonitrile (1.5 equiv), allylpalladium chloride dimer (0.05 equiv), tri-tert-butylphosphonium tetrafluoroborate (0.30 equiv), potassium bicarbonate (3.0 equiv), 2-butanol (5.0 volumes), and 2,2-dimethylbutyric acid (0.30 equiv). The resulting mixture was agitated at about 90° C. for about 4 hours. The reactor contents were cooled to about 50° C., and n-heptane (5.0 volumes) was added. The reactor contents were cooled to about 20° C. over about 1 hour. The slurry was aged for about 30 minutes, then filtered and washed twice with 2-butanol (1.0 volume, then 3.0 volumes). A reactor was charged with the cake, 2-MeTHF (20.0 volumes) and water (10.0 volumes) and mixed to dissolve. The aqueous phase was removed and the organic phase was washed with water (10.0 volumes). The aqueous phase was removed and the organic phase was concentrated to about 5 volumes at about 60° C. The mixture was solvent-swapped to acetone (5.0 volumes) and the contents of reactor were adjusted to about 10° C. and the slurry was aged for about 1 hour. The slurry was filtered and washed with acetone (1.0 volume) and dried at about 50° C. to provide tert-butyl (R)-4-((1-cyanoethyl)amino)-6-(3-cyanopyrrolo[1,2-b]pyridazin-7-yl)nicotinate.

¹H NMR (400 MHZ, DMSO-d₆) δ 8.87 (s, 1H), 8.85 (d, J=2.2 Hz, 1H), 8.65 (d, J=2.2 Hz, 1H), 8.29 (s, 1H), 8.19 (d, J=7.1 Hz, 1H), 7.86 (d, J=4.8 Hz, 1H), 7.11 (d, J=4.8 Hz, 1H), 4.94 (p, J=7.0 Hz, 1H), 1.73 (d, J=6.9 Hz, 3H), 1.57 (s, 9H) ppm.

### Procedure H: Synthesis of (R)-4-((1-cyanoethyl)amino)-6-(3-cyanopyrrolo[1,2-b]pyridazin-7-yl)nicotinic acid methanesulfonate

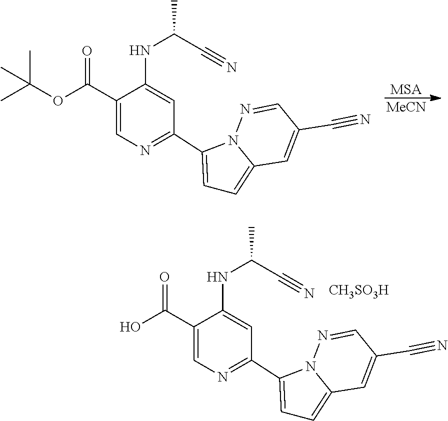

A reactor was charged with tert-butyl (R)-4-((1-cyanoethyl)amino)-6-(3-cyanopyrrolo[1,2-b]pyridazin-7-yl)nicotinate (1.0 equiv, scaling factor) and acetonitrile (5.0 volumes). The mixture was cooled to about 10° C. and methanesulfonic acid (2.0 equiv) was charged. The resulting mixture was agitated at about 65° C. for about 6 hours. The reactor contents were cooled to about 15° C. over about 5 hours. The slurry was aged for about 1 hour and then filtered, and the filtered cake was washed with acetonitrile (5.0 volumes). The cake was dried at about 50° C. to provide (R)-4-((1-cyanoethyl)amino)-6-(3-cyanopyrrolo[1,2-b]pyridazin-7-yl)nicotinic acid methanesulfonate.

¹H NMR (400 MHz, DMSO-d₆): δ 9.37 (d, J=8.0 Hz, 1H), 9.02 (d, J=4.0 Hz, 1H), 8.93 (s, 1H), 8.85 (d, J=4.0 Hz, 1H), 8.18 (d, J=4.0 Hz, 1H), 8.09 (s, 1H), 7.27 (d, J=4.0 Hz, 1H), 5.35 (p, J=4.0 Hz, 1H), 2.38 (s, 3H), 1.79 (d, J=4.0 Hz, 3H) ppm.

### Procedure I: Synthesis of 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide (Compound I)

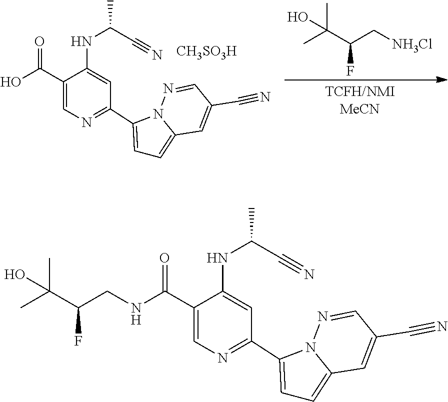

A reactor was charged with (R)-4-((1-cyanoethyl)amino)-6-(3-cyanopyrrolo[1,2-b]pyridazin-7-yl)nicotinic acid methane sulfonate (1.0 equiv, scaling factor), (R)-4-amino-3-fluoro-2-methylbutan-2-ol hydrochloride (1.2 equiv), and acetonitrile (15.0 volumes). The resulting mixture was adjusted to about 0° C. and then 1-methylimidazole (5.0 equiv) was charged. The mixture was aged at about 0° C. for about 30 minutes. A solution of TCFH (1.15 equiv) in acetonitrile (5 volumes) was charged over about 2 hours. The reaction mixture was aged for about 1 hour before adjusting to about 20° C. A separate reactor was charged with water (45.0 volumes) and adjusted to about 50° C. The reaction mixture was charged to the reactor containing water over about 2 hours. The resulting slurry was aged for about 2 hours at about 50° C. before cooling to about 20° C. over about 4 hours. The slurry was aged for about 16 hours, filtered and the filter cake was washed with water (10.0 volumes). The cake was dried under vacuum at about 50° C. provide 4-(((R)-1-cyanoethyl)amino)-6-(3-cyanopyrrolo[1,2-b]pyridazin-7-yl)-N—((R)-2-Fluoro-3-Hydroxy-3-Methylbutyl)nicotinamide.

¹H NMR (400 MHZ, DMSO-d₆): δ 8.89 (t, J=5.8 Hz, 1H), 8.81 (s, 1H), 8.78 (d, J=7.2 Hz, 1H), 8.61 (d, J=2.4 Hz, 1H), 8.22 (s, 1H), 7.85 (d, J=4.4 Hz, 1H), 7.10 (d, J=4.8 Hz, 1H), 4.86 (p, J=6.9 Hz, 1H), 4.40 (ddd, J=49.2, 9.2, 2.0 Hz, 1H), 3.75 (dddd, J=37.2, 14.4, 5.2, 2.0 Hz, 1H), 3.43 (m, 1H), 1.72 (d, J=7.2 Hz, 3H), 1.19 (d, J=1.6 Hz, 3H), 1.18 (d, J=1.2 Hz, 3H) ppm.

¹³C NMR (100 MHZ, DMSO-d₆): δ 167.5, 152.0, 149.7, 149.2, 141.7, 133.8, 130.6, 127.3, 119.7, 118.5, 117.2, 110.1, 106.9, 104.0, 96.9 (d, J=177.8 Hz), 93.5, 69.5 (d, J=19.9 Hz), 39.4 (m), 26.1 (d, J=3.8 Hz), 24.7 (d, J=3.8 Hz), 18.5 ppm.

### Procedure J: Alternate synthesis of 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide (Compound I)

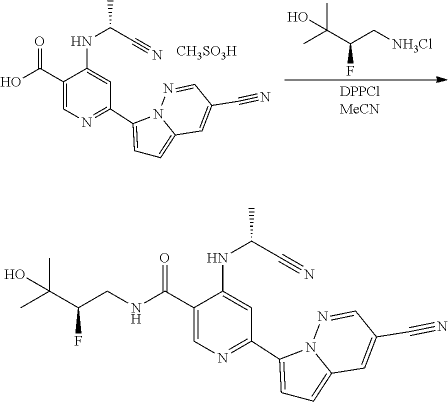

A reactor was charged with (R)-4-((1-cyanoethyl)amino)-6-(3-cyanopyrrolo[1,2-b]pyridazin-7-yl)nicotinic acid methane sulfonate (1.0 equiv, scaling factor) and acetonitrile (15.0 volumes). The resulting slurry was adjusted to about 20° C. and N,N-diisopropylethylamine (5.0 equiv) was added, followed by diphenylphosphinic chloride (1.2 equiv). (R)-4-amino-3-fluoro-2-methylbutan-2-ol hydrochloride (1.2 equiv) was charged as solids, and rinsed forward with acetonitrile (1.0 volumes). The resulting mixture was adjusted to about 0° C. and then 1-methylimidazole (5.0 equiv) was charged. The mixture was aged at about 20° C. for about 1 hour. To this mixture was charged 2-methyltetrahydrofuran (30.0 volumes) and washed three times with about 10% w/w aqueous potassium bicarbonate (15.0 volumes each) and two times with water (15.0 volumes each). The organic layer was azeotropically dried in 2-methyltetrahydrofuran and solvent-swapped to methanol (15.0 volumes) and the reactor contents adjusted to about 45° C. The reactor contents were cooled to about 10° C. over about 3 hours and stirred about 15 hours at about 10° C. The reactor contents were filtered and the cake dried for approximately 21 hours at approximately 55° C. The cake was transferred to a reactor, and ethanol (15.0 volumes) was added. The slurry was agitated at about 50° C. for about 23 hours, then cooled to about 10° C. over about 5 h. The reactor contents were filtered and the reactor and filter cake rinsed forward with ethanol (2.5 volumes). The cake was dried at about 55° C. to give 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide.

¹H NMR (400 MHZ, DMSO-d₆): § 8.89 (t, J=5.8 Hz, 1H), 8.81 (s, 1H), 8.78 (d, J=7.2 Hz, 1H), 8.61 (d, J=2.4 Hz, 1H), 8.22 (s, 1H), 7.85 (d, J=4.4 Hz, 1H), 7.10 (d, J=4.8 Hz, 1H), 4.86 (p, J=6.9 Hz, 1H), 4.40 (ddd, J=49.2, 9.2, 2.0 Hz, 1H), 3.75 (dddd, J=37.2, 14.4, 5.2, 2.0 Hz, 1H), 3.43 (m, 1H), 1.72 (d, J=7.2 Hz, 3H), 1.19 (d, J=1.6 Hz, 3H), 1.18 (d, J=1.2 Hz, 3H) ppm.

¹³C NMR (100 MHZ, DMSO-d₆): δ 167.5, 152.0, 149.7, 149.2, 141.7, 133.8, 130.6, 127.3, 119.7, 118.5, 117.2, 110.1, 106.9, 104.0, 96.9 (d, J=177.8 Hz), 93.5, 69.5 (d, J=19.9 Hz), 39.4, (m), 26.1 (d, J=3.8 Hz), 24.7 (d, J=3.8 Hz), 18.5 ppm.

### Procedure K: Synthesis of (R)-4,6-chloro-N-(2-fluoro-3-hydroxy-3-methylbutyl)nicotinamide

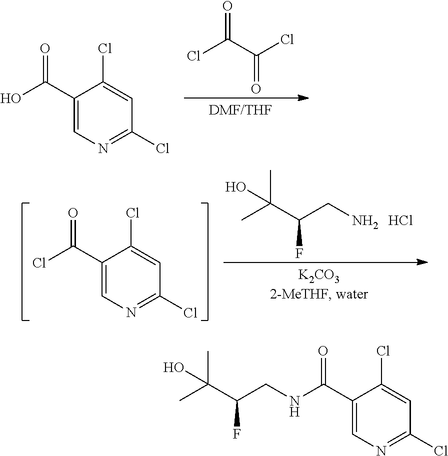

A reactor was charged with 4,6-dichloronicotinic acid (1.1 equiv), N,N-dimethylformamide (0.05 equiv), and tetrahydrofuran (6.0 volumes) and the mixture was cooled to about 10° C. To this mixture was charged oxalyl chloride (1.3 equiv) over about 1 hour and the reaction was agitated at about 10° C. for about 1 hour. A separate reactor was charged with (R)-4-amino-3-fluoro-2-methylbutan-2-ol hydrochloride (1.0 equiv, scaling factor), potassium carbonate (2.5 equiv), 2-methyltetrahydrofuran (8.0 volumes), and water (8.0 volumes) and adjusted to about 10° C. The mixture from the first reactor was charged to the reactor containing (R)-4-amino-3-fluoro-2-methylbutan-2-ol hydrochloride and the reaction was agitated for about 3 hours. The reaction mixture was then adjusted to about 22° C. and agitation was stopped. The lower aqueous layer was removed and the organic was washed twice with 15% w/w sodium chloride in water (5.0 volumes each). The organic layer was azeotropically dried in 2-methyltetrahydrofuran then polish filtered to remove inorganics. After concentrating the organic layer to about 7 volumes, the reaction temperature was adjusted to about 55° C. n-Heptane (3.0 volumes) was then charged over about 1 hour. The reaction temperature was adjusted to about 45° C. and the mixture was aged for about 1 hour before additional n-heptane (13.0 volumes) was charged over about 3 hours. The resulting slurry was aged for about 1 hour at about 45° C. before cooling to about 0° C. over about 8 hours and filtering. The cake was washed twice with 1:3 2-methyltetrahydrofuran:n-heptane (3.0 volumes each). The cake was dried at about 45° C. to provide (R)-4,6-chloro-N-(2-fluoro-3-hydroxy-3-methylbutyl)nicotinamide.

¹H NMR (400 MHZ, DMSO-d₆) δ 8.91 (t, 1H), 8.47 (s, 1H), 7.92 (s, 1H), 4.86 (s, 1H), 4.31 (ddd, J=49.4, 9.4, 2.1 Hz, 1H), 3.77 (m, 1H), 3.34 (m, 1H), 1.16 (dd, J=6.3, 1.7 Hz, 6H) ppm.

### Procedure L: Synthesis of 4-(((R)-1-amino-1-oxopropan-2-yl)amino)-6-chloro-N—((R)-2-fluoro-3-hydroxy-3-methylbutyl)nicotinamide

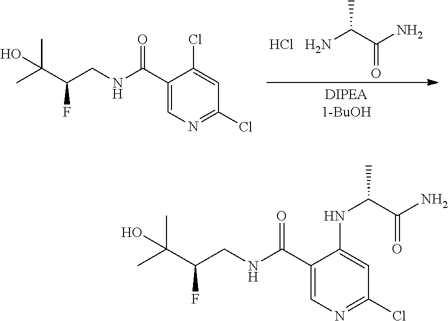

A reactor was charged with (R)-4,6-chloro-N-(2-fluoro-3-hydroxy-3-methylbutyl)nicotinamide (1.0 equiv. scaling factor) and (R)-2-aminopropanamide hydrochloride (2.0 equiv), 1-Butanol (5.0 volumes) and N,N-diisopropylethylamine (4.5 equiv) was charged. The resulting mixture was agitated at about 110° C. for about 18 hrs. The contents of reactor were cooled to about 10° C. and washed twice with 10% w/w potassium bicarbonate in water (5.0 volumes each). The organic phase was transferred to a container and the combined aqueous phases were returned to the reactor and back-extracted with 1-butanol (3.0 volumes). The aqueous phase was removed. The organic phases were combined and washed with 15% w/w sodium chloride in water (5.0 volumes). After concentrating the organic layer to about 3 volumes. 1-butanol (13.0 volumes) was charged. The contents of the reactor were adjusted to about 45° C. and polish filtered, rinsing forward with 1-butanol (2.0 volumes). After concentrating the solution to about 4 volumes, the internal temperature was adjusted to about 75° C. and aged for about 1 hour. The slurry was cooled to about 20° C. over about 5 hours, n-Heptane (10.0 volumes) was charged over about 4 hours, then the slurry was stirred for about 1 hour, then filtered. The cake was washed with a 3:1 mixture of n-heptane: 1-butanol (2.0 volumes) and dried under vacuum at about 50° C. to provide 4-(((R)-1-amino-1-oxopropan-2-yl)amino)-6-chloro-N—((R)-2-fluoro-3-hydroxy-3-methylbutyl)nicotinamide.

¹H NMR (400 MHz, DMSO-d₆) δ 8.71-8.81 (m, 2H), 8.39 (s, 1H), 7.59 (s, 1H), 7.21 (s, 1H), 6.48 (s, 1H), 4.82 (s, 1H), 4.31 (ddd, J=49.4, 9.4, 2.1 Hz, 1H), 4.05 (p. J=6.8 Hz, 1H), 3.53-3.77 (m. 1H), 3.27-3.43 (m, 1H), 1.31 (d, J=6.7 Hz, 3H), 1.14 (dd, J=6.3, 1.7 Hz. 6H) ppm.

### Procedure M: Synthesis of 6-chloro-4-(((R)-1-cyanoethyl)amino)-N—((R)-2-fluoro-3-hydroxy-3-methylbutyl)nicotinamide

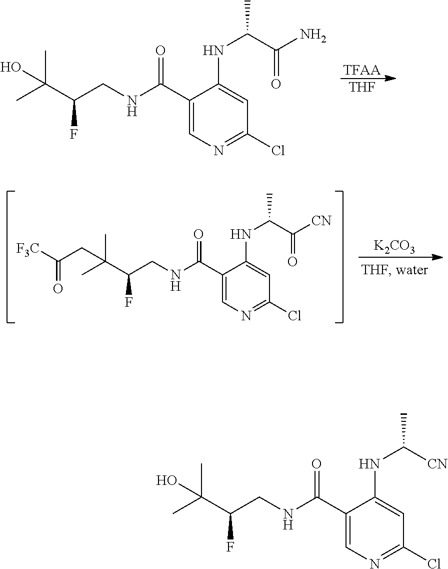

A reactor was charged with 4-(((R)-1-amino-1-oxopropan-2-yl)amino)-6-chloro-N—((R)-2-fluoro-3-hydroxy-3-methylbutyl)nicotinamide (1.0 equiv. scaling factor) and tetrahydrofuran (4.0 volumes). The mixture was cooled to about 0° C. and trifluoroacetic anhydride (2.1 equiv) was charged. The resulting mixture was agitated at about 0° C. for about 1 hour. A separate reactor was charged with potassium carbonate (6.0 equiv) and water (6.0 volumes) and agitated to dissolve. The carbonate solution was then transferred to the reaction mixture and agitated at about 20° C. for about 1 hour, after which isopropyl acetate (8.0 volumes) was charged. The layers were separated and the organic stream was washed twice with water (5.0 volumes each). The resulting organic stream was solvent swapped into isopropanol (2.0 volumes) and heated to about 50° C. to obtain a homogeneous solution, n-Heptane (2.0 volumes) was added and the reactor contents were cooled to about 20° C. over about 6 hours. Additional n-heptane (3.2 volumes) was charged over about 2 hours and the slurry was cooled to about 0° C. over about 2 hours. After aging the slurry for about 5 hours, the slurry was filtered, and the cake was washed with n-heptane (2.0 volumes). The resulting cake was dried at about 50° C. to provide 6-chloro-4-(((R)-1-cyanoethyl)amino)-N—((R)-2-fluoro-3-hydroxy-3-methylbutyl)nicotinamide.

¹H NMR (400 MHZ, DMSO-d₆): δ 8.94 (t, J=4.0 Hz, 1H), 8.69 (d, J=8.0 Hz, 1H), 8.51 (s, 1H), 6.99 (s, 1H), 4.94 (p, J=4.0 Hz, 1H), 4.85 (s, 1H), 4.35 (ddd, J=40.0, 8.0, 4.0 Hz, 1H), 3.70 (dddd, J=40.0, 16.0, 8.0, 4.0 Hz, 1H), 3.39 (tdd, J=16.0, 12.0, 8.0 Hz, 1H), 1.62 (d, J=4.0 Hz, 3H), 1.16 (dd, J=4.0, 8.0 Hz, 6H) ppm.

### Procedure N: Synthesis of 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide (Compound I)

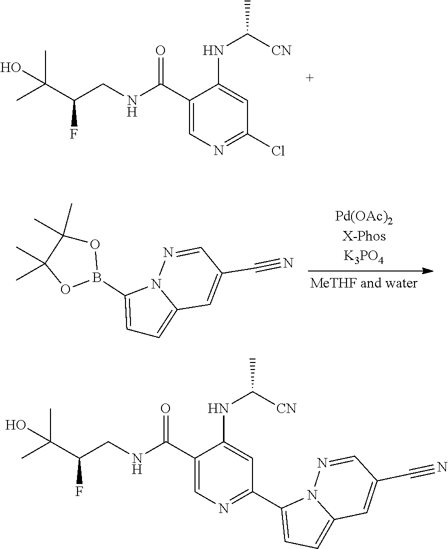 [SMILES: C[C@H](C#N)Nc1cc(Cl)ncc1C(=O)NC[C@@H](F)C(C)(C)O ; CC1(C)OB(c2ccc3cc(C#N)cnn23)OC1(C)C ; C[C@H](C#N)Nc1cc(-c2ccc3cc(C#N)cnn23)ncc1C(=O)NC[C@@H](F)C(C)(C)O]

A reactor was charged with 6-chloro-4-(((R)-1-cyanoethyl)amino)-N—((R)-2-fluoro-3-hydroxy-3-methylbutyl)nicotinamide (1.0 equiv, scaling factor), 7-(4,4,5,5-tetramethyl-1,3,2-dioxaborolan-2-yl)pyrrolo[1,2-b]pyridazine-3-carbonitrile (1.7 equiv), XPhos (0.05 equiv), palladium acetate (0.025 equiv) and 2-methyltetrahydrofuran (17.0 volumes). The resulting mixture was agitated and a solution of potassium phosphate tribasic (2.0 equiv) in water (2.5 volumes) was added to the mixture. The reaction mixture was agitated and heated to about 75° C. for about 4 hours, then cooled to about 40° C. To the reaction mixture was charged diatomaceous earth (200% w/w), trithiocyanuric acid trisodium hydrate (0.3 equiv), Darco G-60 (50% w/w), and 2-methyltetrahydrofuran (21.0 volumes). The resulting mixture was agitated for about 17 hours at about 40° C., then filtered. The reactor and filter cake were rinsed three times with 2-methyltetrahydrofuran (10.0 volumes each). The combined filtrates were washed three times with water (10.0 volumes each) at about 50° C., then the organic stream was concentrated to about 10 volumes. The reactor was charged with 2-methyltetrahydrofuran (20.0 volumes). The resulting solution was passed through a polish filter, then concentrated under reduced pressure to about 10 volumes. The reaction mixture was solvent-swapped into methanol (15.0 volumes) and the reactor contents adjusted to about 45° C. The reactor contents were cooled to about 10° C. over about 3 hours and stirred about 15 hours at about 10° C. The reactor contents were filtered and the cake dried for approximately 21 hours at approximately 55° C. The cake was transferred to a reactor, and ethanol (15.0 volumes) was added. The slurry was agitated at about 50° C. for about 23 hours, then cooled to about 10° C. over about 5 h. The reactor contents were filtered and the reactor and filter cake rinsed forward with ethanol (2.5 volumes). Toluene (10 volumes) was added to the filter and the mixture agitated for about 1 hour, then filtered. The cake was then rinsed with toluene (5.0 volumes) and dried at about 55° C. to give 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide.

¹H NMR (400 MHz, DMSO-d₆): δ 8.89 (t. J=5.8 Hz, 1H), 8.81 (s, 1H), 8.78 (d, J=7.2 Hz, 1H). 8.61 (d, J=2.4 Hz, 1H), 8.22 (s, 1H), 7.85 (d, J=4.4 Hz, 1H), 7.10 (d, J=4.8 Hz, 1H), 4.86 (p. J=6.9 Hz. 1H), 4.40 (ddd, J=49.2, 9.2, 2.0 Hz, 1H), 3.75 (dddd, J=37.2, 14.4, 5.2, 2.0 Hz, 1H), 3.43 (m, 1H). 1.72 (d, J=7.2 Hz, 3H), 1.19 (d, J=1.6 Hz, 3H), 1.18 (d, J=1.2 Hz, 3H) ppm.

¹³C NMR (100 MHz, DMSO-d₆): δ 167.5, 152.0, 149.7, 149.2, 141.7, 133.8, 130.6, 127.3, 119.7, 118.5, 117.2, 110.1, 106.9, 104.0, 96.9 (d, J=177.8 Hz), 93.5, 69.5 (d, J=19.9 Hz), 39.4, (m), 26.1 (d, J=3.8 Hz), 24.7 (d, J=3.8 Hz), 18.5 ppm.

### Procedure O: Coupling Reaction of 4-(((R)-1-amino-1-oxopropan-2-yl)amino)-6-chloro-N—((R)-2-fluoro-3-hydroxy-3-methylbutyl)nicotinamide with 7-(4,4,5,5-tetramethyl-1,3,2-dioxaborolan-2-yl)pyrrolo[1,2-b]pyridazine-3-carbonitrile to 4-(((R)-1-amino-1-oxopropan-2-yl)amino)-6-(3-cyanopyrrolo[1,2-b]pyridazin-7-yl)-N—((R)-2-fluoro-3-hydroxy-3-methylbutyl)nicotinamide

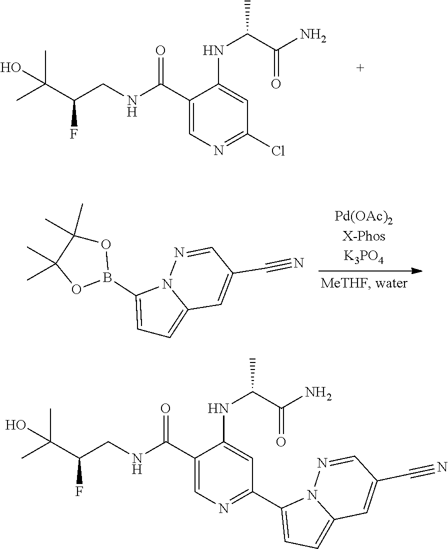 [SMILES: C[C@@H](Nc1cc(Cl)ncc1C(=O)NC[C@@H](F)C(C)(C)O)C(N)=O ; CC1(C)OB(c2ccc3cc(C#N)cnn23)OC1(C)C ; C[C@@H](Nc1cc(-c2ccc3cc(C#N)cnn23)ncc1C(=O)NC[C@@H](F)C(C)(C)O)C(N)=O]

A reactor was charged with 4-(((R)-1-amino-1-oxopropan-2-yl)amino)-6-chloro-N—((R)-2-fluoro-3-hydroxy-3-methylbutyl)nicotinamide (1.0 equiv. scaling factor), 7-(4,4,5,5-tetramethyl-1,3,2-dioxaborolan-2-yl)pyrrolo[1,2-b]pyridazine-3-carbonitrile (1.6 equiv). XPhos (0.05 equiv), palladium acetate (0.025 equiv) and 2-methyltetrahydrofuran (17.0 volumes). The resulting mixture was agitated and a solution of potassium phosphate tribasic (2.0 equiv) in water (2.5 volumes) was added to the mixture. The reaction mixture was agitated and heated to about 75° C. for about 4 hours, then cooled to about 40° C. The resulting slurry was filtered. The cake was washed with toluene (9.0 volumes), acetonitrile (5.0 volumes) and aqueous citric acid solution (10% w/w), and dried at about 40° C. The solids were charged to a reactor and slurried in N-methyl-2-pyrrolidinone (5.0 volumes) at about 40° C. The slurry was filtered and washed with N-methyl-2-pyrrolidinone (2.0 volumes) and the resulting organic stream was concentrated to about 5 volumes. The mixture was charged to a reactor, followed by diatomaceous earth (200% w/w), trithiocyanuric acid trisodium hydrate (0.3 equiv), and Darco G-60 (50% w/w) and mixed at about 40° C. for about 2 h. The mixture was filtered and the filtrate concentrated to about 1 volume. Ethanol (18.0 volumes) was added at about 40° C. and aged at about 50° C. for about 2 h. Water (18.0 volumes) was added at about 35° C. and aged for about 2 h and cooled to about 20° C. over about 3 h. The slurry was filtered and washed with water (5.0 volumes). The reactor contents were filtered and the cake dried for approximately 21 hours at approximately 40° C. to give 4-(((R)-1-amino-1-oxopropan-2-yl)amino)-6-(3-cyanopyrrolo[1,2-b]pyridazin-7-yl)-N—((R)-2-fluoro-3-hydroxy-3-methylbutyl)nicotinamide.

1H NMR (400 MHZ. Methanol-d₄) δ 8.76 (d, J=2.2 Hz, 1H), 8.69-8.63 (m, 2H), 8.05 (d, J=4.8 Hz, 1H), 7.88 (s, 1H), 7.22 (d, J=5.1 Hz, 1H), 4.91 (t, J=7.6 Hz, 2H), 4.73 (dd, J=8.2, 4.4 Hz, 2H), 4.52-4.28 (m, 3H), 3.80-3.40 (m, 5H), 2.76 (dd, J=13.9, 7.0 Hz, 2H), 2.24 (s, 2H), 1.29 (d, J=1.7 Hz, 6H).

### Procedure P: Amide Dehydration Reaction of 4-(((R)-1-amino-1-oxopropan-2-yl)amino)-6-(3-cyanopyrrolo[1,2-b]pyridazin-7-yl)-N—((R)-2-fluoro-3-hydroxy-3-methylbutyl)nicotinamide to 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide (Compound I)

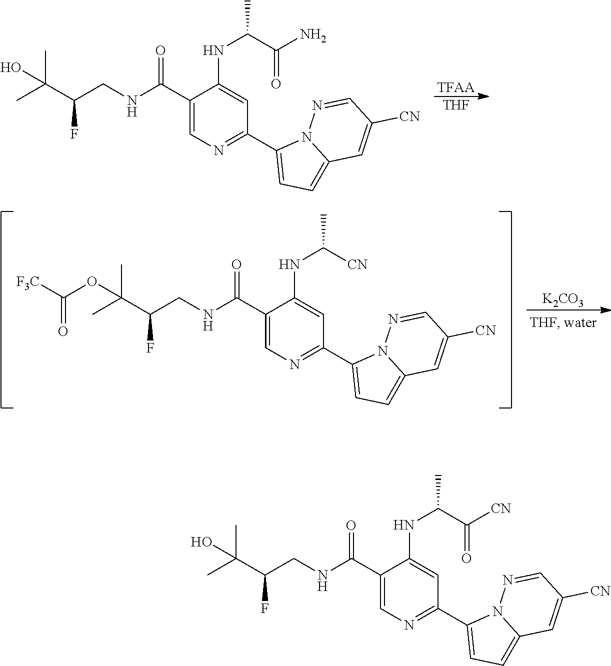

A reactor was charged with 4-(((R)-1-amino-1-oxopropan-2-yl)amino)-6-(3-cyanopyrrolo[1,2-b]pyridazin-7-yl)-N—((R)-2-fluoro-3-hydroxy-3-methylbutyl)nicotinamide and THF (17.0 volumes). Pyridine (5.0 equiv) was charged and the mixture was cooled to about 0° C. Trifluoroacetic anhydride (2.9 equiv) was charged and resulting mixture was agitated at about 0° C. for about 1 hour. A sodium carbonate solution (1M, 15 volumes) was charged over about 1 h and the mixture warmed to about 20° C. over about 1 h. Water (20.0 volumes) and ethyl acetate (20.0 volumes) were charged and the mixture filtered over celite and rinsed with ethyl acetate (20.0 volumes). The layers were separated and the aqueous stream extracted with 2-methyltetrahydrofuran (20.0 volumes). The organic streams were combined and washed with a saturated aqueous ammonium chloride solution (20.0 volumes). The resulting organic stream was solvent swapped into ethanol (15.0 volumes) and n-heptane (50.0 volumes) was added over about 1 h at about 20° C. After aging the slurry for about 3 hours, the slurry was filtered, and the cake was washed with n-heptane (2.0 volumes). The resulting cake was dried at about 50° C. to provide 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide (Compound I).

¹H NMR (400 MHZ, DMSO-d₆): δ 8.89 (t, J=5.8 Hz, 1H), 8.81 (s, 1H), 8.78 (d, J=7.2 Hz, 1H), 8.61 (d, J=2.4 Hz, 1H), 8.22 (s, 1H), 7.85 (d, J=4.4 Hz, 1H), 7.10 (d, J=4.8 Hz, 1H), 4.86 (p, J=6.9 Hz, 1H), 4.40 (ddd, J=49.2, 9.2, 2.0 Hz, 1H), 3.75 (dddd, J=37.2, 14.4, 5.2, 2.0 Hz, 1H), 3.43 (m, 1H), 1.72 (d, J=7.2 Hz, 3H), 1.19 (d, J=1.6 Hz, 3H), 1.18 (d, J=1.2 Hz, 3H) ppm.

¹³C NMR (100 MHZ, DMSO-d₆): δ 167.5, 152.0, 149.7, 149.2, 141.7, 133.8, 130.6, 127.3, 119.7, 118.5, 117.2, 110.1, 106.9, 104.0, 96.9 (d, J=177.8 Hz), 93.5, 69.5 (d, J=19.9 Hz), 39.4, (m), 26.1 (d, J=3.8 Hz), 24.7 (d, J=3.8 Hz), 18.5 ppm.

### Procedure Q: Synthesis of 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide citrate (Compound I Citric Acid Salt)

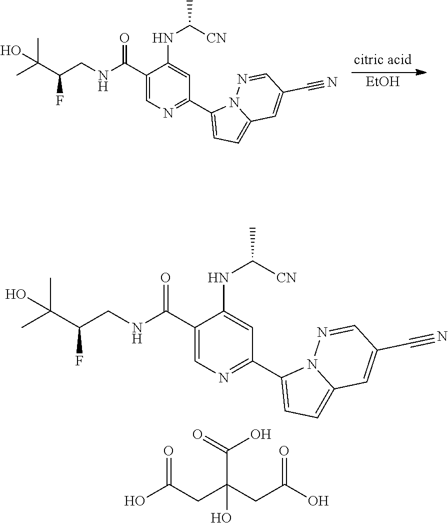

A reactor was charged with 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide (Compound I, 1.0 equiv, scaling factor) and ethanol (10.0 volumes). The resulting mixture was heated to about 50° C. and agitated. To a separate reactor was charged citric acid (1.5 equiv) and ethanol (20.0 volumes). The citric acid solution was agitated and heated to about 35° C. for about 30 minutes to dissolve and was charged to the reactor containing Compound I. The reaction mixture was agitated at about 50° C. for about 18 hours, then cooled to about 20° C. over about 3 hours. The reactor contents were filtered and the reactor and filter cake were rinsed forward with ethanol (5.0 volumes). The cake was dried at about 40° C. to give 4-(((R)-1-cyanoethyl)amino)-6-(3-cyanopyrrolo[1,2-b]pyridazin-7-yl)-N—((R)-2-fluoro-3-hydroxy-3-methylbutyl)nicotinamide citrate.

¹H NMR (400 MHZ, DMSO-d₆) δ 8.91 (t, J=5.6 Hz, 1H), 8.87 (d, J=2.2 Hz, 1H), 8.83 (s, 1H), 8.78 (d, J=7.1 Hz, 1H), 8.66 (d, J=2.2 Hz, 1H), 8.25 (s, 1H), 7.88 (d, J=4.8 Hz, 1H), 7.14 (d, J=4.8 Hz, 1H), 4.89 (dd, J=14.4, 7.4 Hz, 2H), 4.39 (ddd, J=49.2, 9.3, 2.1 Hz, 1H), 3.75 (dddd, J=37.3, 14.5, 5.3, 2.2 Hz, 1H), 3.56-3.37 (m, 1H), 2.77 (d, J=15.4 Hz, 2H), 2.66 (d, J=15.4 Hz, 2H), 2.51 (p, J=1.9 Hz, 3H), 1.72 (d, J=7.0 Hz, 3H), 1.18 (dd, J=5.3, 1.6 Hz, 6H) ppm.

¹³C NMR (101 MHz, DMSO-d₆) δ 174.5, 171.2, 167.4, 152.0, 149.7, 149.2, 141.8, 134.0, 130.5, 127.4, 119.7, 118.5, 117.2, 110.2, 107.0, 104.1, 96.9 (d, J=177.8 Hz), 93.6, 72.4, 69.5 (d, J=19.9 Hz), 42.7, 38.6, 26.1 (d, J=3.8 Hz), 24.7 (d, J=3.8 Hz), 18.5 ppm.

Unless otherwise defined, all technical and scientific terms used herein have the same meaning as commonly understood by one of ordinary skill in the art to which this disclosure belongs.

The embodiments illustratively described herein may suitably be practiced in the absence of any element or elements, limitation or limitations, not specifically disclosed herein. Thus, for example, the terms “comprising.” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the embodiments claimed.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A process for preparing Compound I, or a salt thereof: 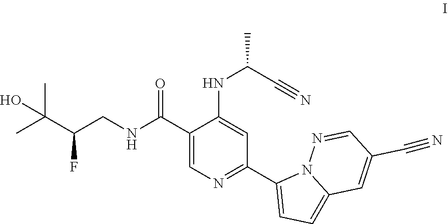 [SMILES: C[C@H](C#N)Nc1cc(-c2ccc3cc(C#N)cnn23)ncc1C(=O)NC[C@@H](F)C(C)(C)O]
comprising contacting Compound II, or a salt thereof; 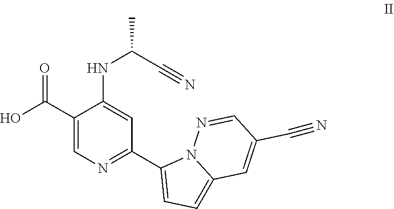 [SMILES: C[C@H](C#N)Nc1cc(-c2ccc3cc(C#N)cnn23)ncc1C(=O)O]
with Compound III, or a salt thereof: 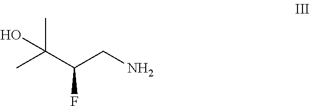
under conditions suitable to provide Compound I.

2. The process of claim 1, wherein the process further comprises contacting Compound I with citric acid to provide Compound I citric acid salt:

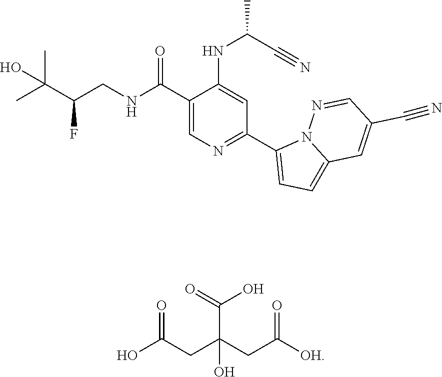 [SMILES: C[C@H](C#N)Nc1cc(-c2ccc3cc(C#N)cnn23)ncc1C(=O)NC[C@@H](F)C(C)(C)O ; O=C(O)CC(O)(CC(=O)O)C(=O)O]

3. A citric acid salt of Compound I:

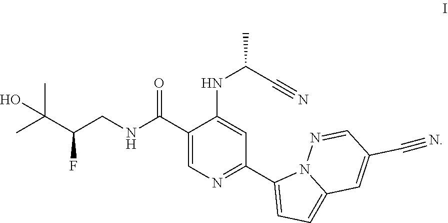 [SMILES: C[C@H](C#N)Nc1cc(-c2ccc3cc(C#N)cnn23)ncc1C(=O)NC[C@@H](F)C(C)(C)O]

4. A methanesulfonic acid salt of Compound II:

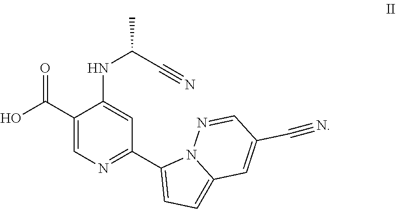 [SMILES: C[C@H](C#N)Nc1cc(-c2ccc3cc(C#N)cnn23)ncc1C(=O)O]

5. A process for preparing Compound II, or a salt thereof: 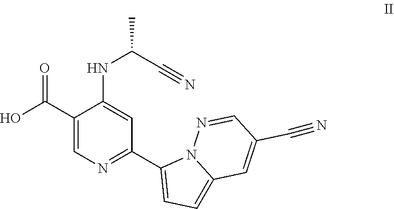 [SMILES: C[C@H](C#N)Nc1cc(-c2ccc3cc(C#N)cnn23)ncc1C(=O)O]
comprising contacting a compound of Formula IV, or a salt thereof; 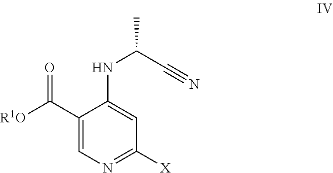
with a compound of Formula V, or a salt thereof: 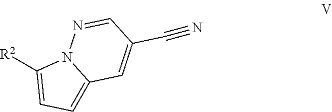
under conditions suitable to provide Compound I; wherein:
X is halo;
R¹ is a protecting group;
R² is hydrogen or —B(OR³)₂; and
each R³ is independently hydrogen or C₁₋₆ alkyl, or two R³ cyclize to form a cyclic boronate ester.

6. The process of claim 5, wherein the conditions further comprise a deprotection step.

7. The process of claim 6, wherein the deprotection step comprises converting a compound of Formula IIA to Compound II:

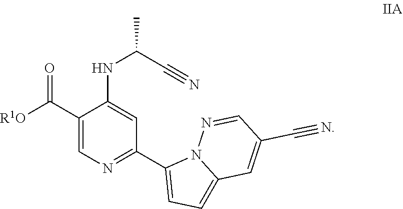

8. The process of claim 5, wherein R¹ is methyl, ethyl, n-propyl, isopropyl, n-butyl, isobutyl, sec-butyl, tert-butyl, or benzyl.

9. The process of claim 8, wherein the deprotection step comprises methane sulfonic acid.

10. The process of claim 9, wherein the process provides Compound II methanesulfonic acid salt:

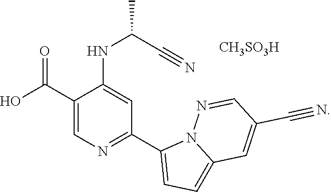 [SMILES: C[C@H](C#N)Nc1cc(-c2ccc3cc(C#N)cnn23)ncc1C(=O)O ; "CH3SO3H"]

11. The process of claim 5, wherein R² is —B(OR³)₂.

12. The process of claim 5, wherein R² is

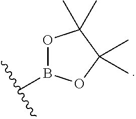 [SMILES: *B1OC(C)(C)C(C)(C)O1]

13. The process of claim 5, wherein the conditions comprise a catalyst, a ligand, and a base.

14. The process of claim 13, wherein the catalyst is a palladium(II) or palladium(0) salt.

15. The process of claim 13, wherein the ligand is 2-dicyclohexylphosphino-2′,4′,6′-triisopropylbiphenyl (XPhos), 2-dicyclohexylphosphino-2′,6′-diisopropoxybiphenyl (RuPhos), a dialkylbiaryl phosphine ligand, a monodentate phosphine ligand, or a bidentate phosphine ligand.

16. The process of claim 13, wherein the base is an inorganic base, an alkoxide base, a tertiary amine, or a nitrogen-containing heteroaryl base.

17. The process of claim 5, wherein R² is hydrogen.

18. The process of claim 17, wherein the conditions comprise a catalyst, a ligand, and an acid.

19. The process of claim 18, wherein the catalyst is a palladium catalyst or a palladium precatalyst.

20. The process of claim 18, wherein the ligand is a monodentate phosphine or a bidentate phosphine.

21. The process of claim 18, wherein the acid is a carboxylic acid or a sulfonic acid.

22. A process for preparing Compound I, or a salt thereof: 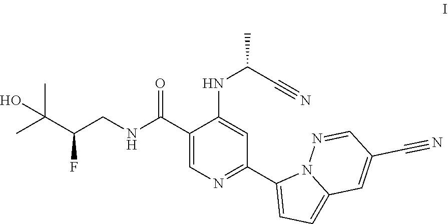 [SMILES: C[C@H](C#N)Nc1cc(-c2ccc3cc(C#N)cnn23)ncc1C(=O)NC[C@@H](F)C(C)(C)O]
comprising contacting compound of Formula VI, or a salt thereof: 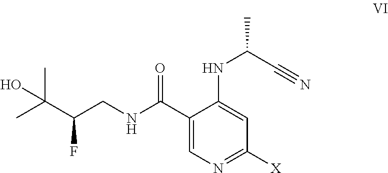
with a compound of Formula V, or a salt thereof: 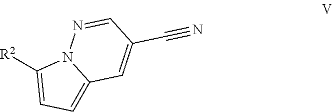
under conditions suitable to provide Compound I; provided the conditions do not comprise microwave radiation; wherein:
X is halo;
R² is hydrogen or —B(OR³)₂; and
each R³ is independently hydrogen or C₁₋₆ alkyl, or two R³ cyclize to form a cyclic boronate ester.

23. The process of claim 22, wherein the process further comprises contacting Compound I with citric acid to provide Compound I citric acid salt:

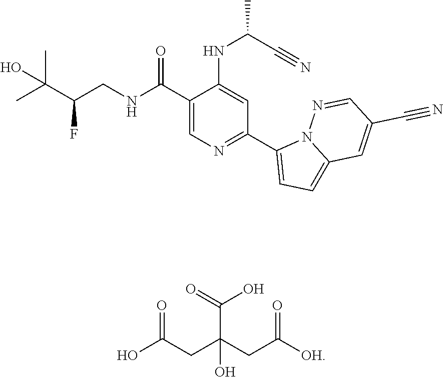 [SMILES: C[C@H](C#N)Nc1cc(-c2ccc3cc(C#N)cnn23)ncc1C(=O)NC[C@@H](F)C(C)(C)O ; O=C(O)CC(O)(CC(=O)O)C(=O)O]

24. The process of claim 22, wherein the conditions comprise a catalyst, a ligand, and a base.

25. The process of claim 22, wherein the conditions comprise palladium(II) acetate, 2-dicyclohexylphosphino-2′,4′,6′-triisopropylbiphenyl (XPhos), and potassium phosphate tribasic.

26. The process of claim 22, wherein the conditions comprise 2-methyltetrahydrofuran and water at a temperature of from about 70° C. to 80° C.

27. A process for preparing a compound of Formula VI, or a salt thereof: 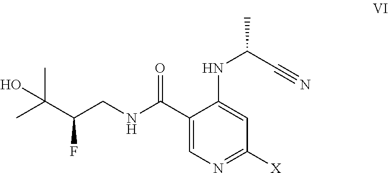
comprising contacting a compound of Formula VII, or a salt thereof, 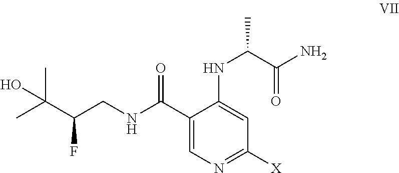
with trifluoroacetic anhydride in the absence of an organic base, under conditions suitable to provide a compound of Formula VIII: 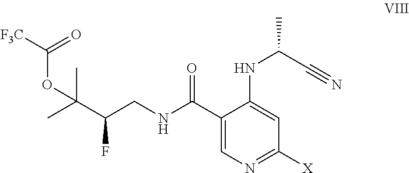
wherein X is halo; and hydrolyzing a compound of Formula VIII to provide a compound of Formula VI.

28. A process for preparing a compound of Formula VI, or a salt thereof: 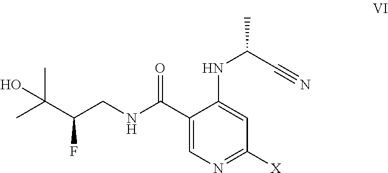
comprising contacting a compound of Formula VII, or a salt thereof; 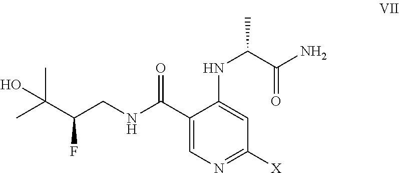
with a dehydrating agent under conditions suitable to provide a compound of Formula VI; wherein X is halo; provided that the dehydrating agent is other than trifluoroacetic anhydride.

29. The process of claim 28, wherein the dehydrating agent is cyanuric chloride, a carboxylic acid anhydride (such as acetic anhydride), a sulfonic acid anhydride (such as trifluoromethanesulfonic anhydride), an alkyl chloroformate (such as ethyl chloroformate), phosphorus oxychloride, or phosphorus pentoxide.

30. The process of claim 28, wherein the process further comprises a hydrolyzing step.

31. The process of claim 30, wherein the hydrolyzing comprises an inorganic base or an alkoxide base.

32. The process of claim 28, wherein the conditions comprise a base.

33. The process of claim 32, wherein the base is an amine base.

34. A process for preparing a compound of Formula IX, or a salt thereof: 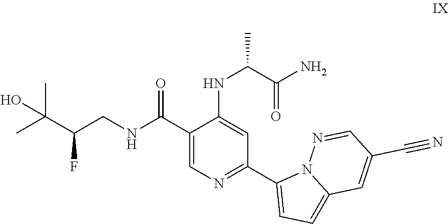 [SMILES: C[C@@H](Nc1cc(-c2ccc3cc(C#N)cnn23)ncc1C(=O)NC[C@@H](F)C(C)(C)O)C(N)=O]
comprising contacting a compound of Formula VII, or a salt thereof; 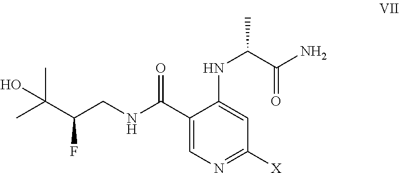
with a compound of Formula V, or a salt thereof: 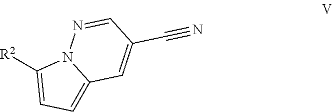
under conditions suitable to provide Compound IX; wherein:
X is halo;
R² is hydrogen or —B(OR³)₂; and
each R³ is independently hydrogen or C₁₋₆ alkyl, or two R³ cyclize to form a cyclic boronate ester.

35. The process of claim 34, wherein the compound of Formula V is represented by a compound of Formula VA:

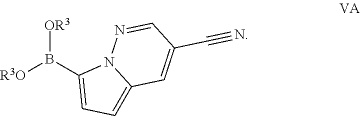

36. The process of claim 5, wherein X is chloro.

37. A process for preparing Compound I, or a salt thereof: 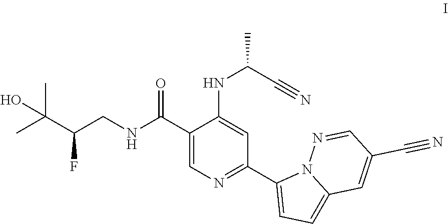 [SMILES: C[C@H](C#N)Nc1cc(-c2ccc3cc(C#N)cnn23)ncc1C(=O)NC[C@@H](F)C(C)(C)O]
comprising contacting a compound of Formula IX, or a salt thereof; 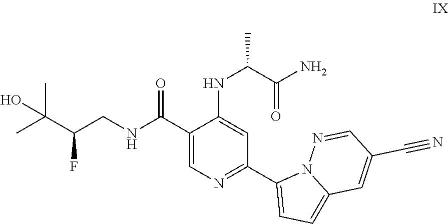 [SMILES: C[C@@H](Nc1cc(-c2ccc3cc(C#N)cnn23)ncc1C(=O)NC[C@@H](F)C(C)(C)O)C(N)=O]
under conditions suitable to provide Compound I.

38. The process of claim 37, wherein the conditions comprise a dehydrating agent, and optional hydrolysis step.

39. A compound, or a salt thereof, selected from: [chemical structure]
